(19) United States
(12) Patent Application Publication
(10) Pub. No.: US 2026/0317349 A1
(43) Pub. Date: Oct. 15, 2026

(54) MACROCYCLIC CHELATING AGENT CAPABLE OF SITE-SPECIFIC MODIFICATION, RADIONUCLIDE PROBE, AND USE THEREOF

(71) Applicant: SHANGHAI INSTITUTE OF MATERIA MEDICA, CHINESE ACADEMY OF SCIENCES, Shanghai (CN)

(72) Inventors: Zhen CHENG, Shanghai (CN); Chunrong QU, Shanghai (CN); Renda LI, Shanghai (CN)

(21) Appl. No.: 19/478,929

(22) PCT Filed: Apr. 19, 2024
(86) PCT No.: PCT/CN2024/088743

§ 371 Date: Dec. 26, 2025

(30) Foreign Application Priority Data
Apr. 27, 2023 (CN) 202310475922.7

(51) Int. Cl. C07F 7/18; A61K 31/555; A61K 31/675; A61K 31/695; A61K 47/55; A61K 51/04; A61K 51/08; C07B 59/00; C07D 471/18; C07F 9/6561

(52) U.S. Cl. CPC C07F 7/1804; A61K 31/555; A61K 31/675; A61K 31/695; A61K 47/55; A61K 51/0482; A61K 51/088; C07B 59/002; C07B 59/004; C07B 59/008; C07D 471/18; C07F 9/6561; C07B 2200/05

(57) ABSTRACT

The present invention relates to a macrocyclic chelating agent capable of site-specific modification, a nuclide probe, and a use thereof, and in particular to a compound as represented by formula I, or a precursor compound, isotope compound, salt, or hydrate thereof. Compared with common macrocyclic chelating agents, the macrocyclic chelating agent of the present application can be used for site-specific modification of various target molecules at R₄ site, comprising antibodies, polypeptides, sugars, nucleic acids, small molecule drugs, optical dyes, organic nanomaterials or fragments or derivatives thereof, and the reaction is more accurate and efficient. The macrocyclic chelating agent can be chelated with radionuclides for imaging such as PET, SPECT, MRI, etc., or for research on the application of radionuclide therapy.

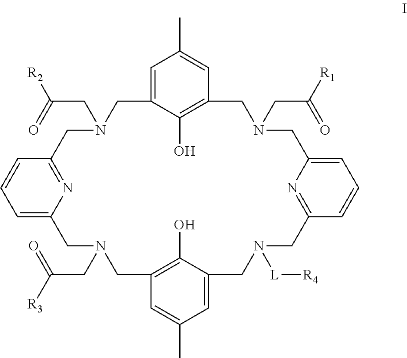

### TECHNICAL FIELD

This invention belongs to the technical field of chelating agent preparation. Specifically, it relates to a novel macrocyclic chelating agent capable of site-specific modification, a nuclide probe based on the macrocyclic chelating agent capable of site-specific modification, and their use in the preparation of molecular imaging diagnostic agents/tumor therapeutic drugs.

### BACKGROUND ART

Nuclear imaging techniques primarily include Positron Emission Tomography (PET) and Single-Photon Emission Computed Tomography (SPECT). By combining with high-resolution Computed Tomography (CT), PET or SPECT can display the molecular imaging characteristics of deep tissues while simultaneously providing high-resolution visualization of anatomical structures of the tissue, and is now widely used in clinical practice. Typically, a radioactive probe requires the target molecule to be coupled with a chelating agent and chelate a radioactive nuclide before being used for nuclear imaging.

Currently used chelating agents typically have several identical functional groups that can be used for both coordination and conjugating with target molecules, such as DOTA and NOTA, which have 4 and 3 carboxyl groups respectively. This leads to increased difficulty in connecting and purifying when conjugating them with the target molecules. On the other hand, although radioactive nuclides are diverse, current chelating agents can only chelate one or a few radioactive nuclides specifically. For example, DOTA can chelate ⁶⁸Ga and ¹⁷⁷Lu, but NOTA and DFO need to be selected respectively for chelating Al[¹⁸F] and ⁸⁹Zr. Currently, there is a need for a universal macrocyclic chelating agent.

### SUMMARY OF THE INVENTION

The invention aims to provide a macrocyclic chelating agent capable of site-specific modification, a nuclide probe, and their uses in the preparation of molecular imaging diagnostic agents/tumor therapeutic drugs. The novel macrocyclic chelating agent of the invention has a single conjugating site, allowing for conjugation of various target molecules at the same site through different groups. Simultaneously, it can broadly chelate various nuclides, showing great promise in the diagnosis and treatment of nuclear imaging.

In order to achieve the above-mentioned aims of the present invention, the following technical solution is adopted.

In a first aspect, the invention provides a compound of formula I, or a precursor compound, isotopic compound, salt, or hydrate thereof,

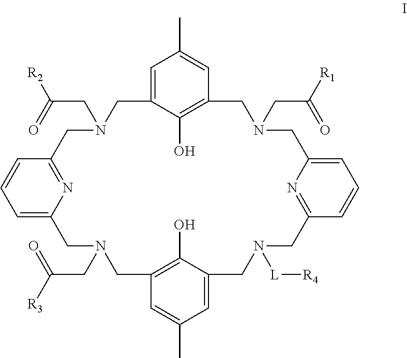

wherein,
R₁, R₂ and R₃ are each independently selected from —OH, —ORₐ, oxygen-containing long-chain polymer group or derivative groups thereof;
Rₐ is selected from substituted or unsubstituted C₁-C₁₂ alkyl, substituted or unsubstituted C₁-C₁₂ silyl, substituted or unsubstituted C₃-C₁₆ cycloalkyl, substituted or unsubstituted 3- to 16-membered heterocyclyl, substituted or unsubstituted C₆-C₁₆ aryl, and substituted or unsubstituted 5- to 16-membered heteroaryl, wherein the substituted refers to being substituted by one or more substituents selected from halogen, C₁-C₆ alkyl, C₁-C₆ alkoxy, oxo, and amino;
L is a linker;
R₄ is an active group capable of reacting (such as conjugating) with a target molecule.

In a preferred embodiment, R₁, R₂, and R₃ are each independently selected from —OH, —ORₐ, PEG or derivative groups thereof; Rₐ is selected from substituted or unsubstituted C₁-C₆ alkyl, substituted or unsubstituted C₁-C₆ silyl, substituted or unsubstituted C₆-C₁₂ aryl, and substituted or unsubstituted 5-10 heteroaryl.

Specifically, Rₐ is selected from tert-butyl, trimethylsilyl (Si(CH₃)₃), benzyl, and N-succinimidyl.

Specifically, the PEG or derivative groups thereof are selected from

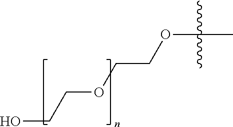

(n is an integer selected from 0 to 10), and

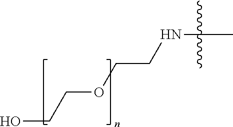

(n is an integer selected from 0 to 10).

In a preferred embodiment, R₄ is any one selected from C₂-C₆ alkynyl, azido, halogen, formyl, hydroxyl, amino, carboxyl, sulfo, phosphate, mercapto, sulfinamido, isothiocyanato (—N═C═S), pentafluorophenyl, maleimido, and N-succinimidyl.

In a preferred embodiment, L is selected from C₁-C₁₂ alkylene, divalent long-chain polymer groups or derivative groups thereof, divalent amino acid groups and derivative groups thereof, divalent polypeptide groups, and bifunctional linkers; preferably is selected from C₁-C₁₂ alkylene, divalent PEG or derivative groups thereof, divalent L or D amino acids and derivative groups thereof, divalent polypeptide groups, and bifunctional linkers.

Preferably, the divalent PEG or derivative groups thereof are selected from

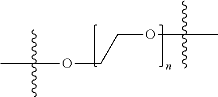

(n is an integer selected from 0 to 10) and

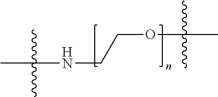

(n is an integer selected from 0 to 10).

Preferably, the bifunctional linker is selected from the following structures:

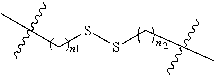

(n1 and n2 are each an integer from 0 to 10),

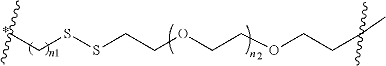

(n1 and n2 are each an integer selected from 0 to 5),

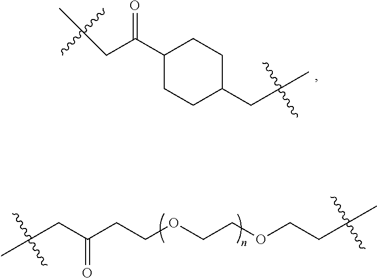

(n is an integer from 0 to 5),

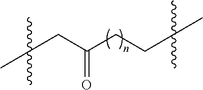

(n is an integer from 0 to 10),

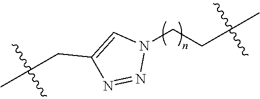

(n is an integer from 0 to 10),

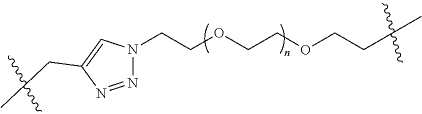

(n is an integer selected from 0 to 5),

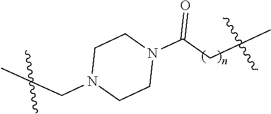

(n is an integer selected from 0 to 5),

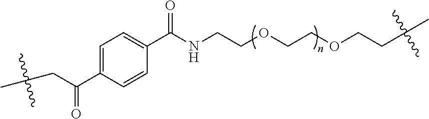

(n is an integer from 0 to 5),

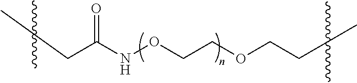

(n is an integer from 0 to 5),

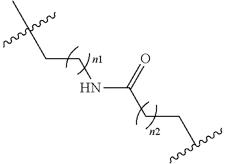

(n1 and n2 are each an integer selected from 0 to 5).

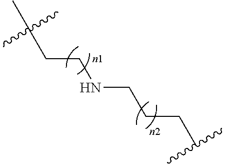

(n1 and n2 are each an integer from 0 to 5),

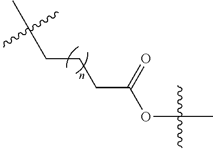

(n is an integer from 0 to 5).

In a preferred embodiment, the compound of formula I is selected from the following compounds:

| No. | Chemical Structure |
|---|---|
| I-1 | 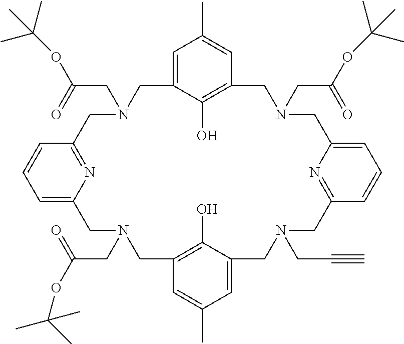 [SMILES: C#CCN1Cc2cccc(n2)CN(CC(=O)OC(C)(C)C)Cc2cc(C)cc(c2O)CN(CC(=O)OC(C)(C)C)Cc2cccc(n2)CN(CC(=O)OC(C)(C)C)Cc2cc(C)cc(c2O)C1] |
| I-2 | 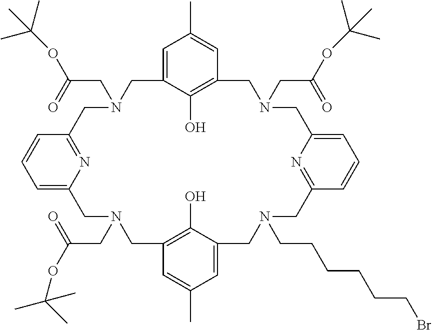 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCCBr)C2] |
| I-3 | 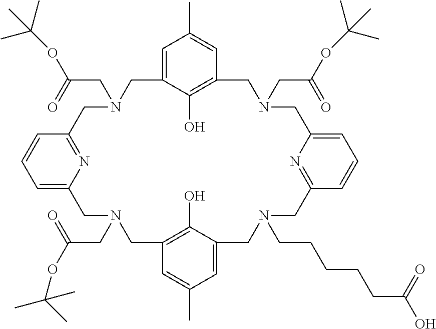 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCC(=O)O)C2] |
| I-4 | 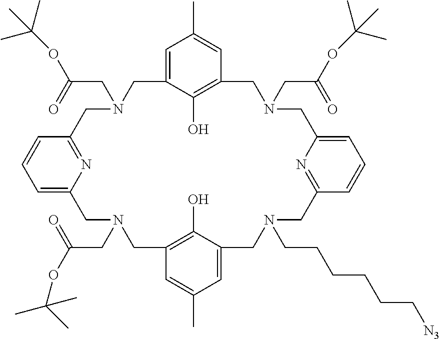 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCCN=[N+]=[N-])C2] |
| I-5 | 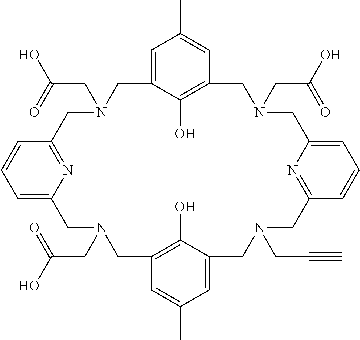 [SMILES: C#CCN1Cc2cccc(n2)CN(CC(=O)O)Cc2cc(C)cc(c2O)CN(CC(=O)O)Cc2cccc(n2)CN(CC(=O)O)Cc2cc(C)cc(c2O)C1] |
| I-6 | 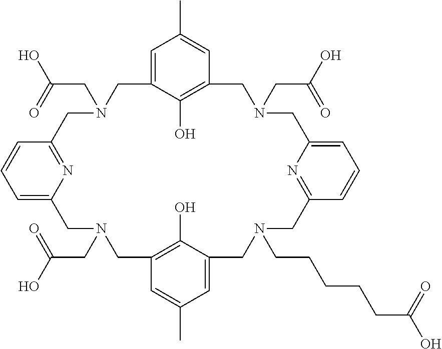 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCC(=O)O)C2] |
| I-7 | 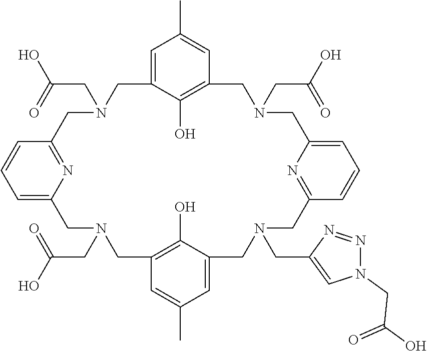 [SMILES: Cc1cc2c(O)c(c1)CN(CC(=O)O)Cc1cccc(n1)CN(Cc1cn(CC(=O)O)nn1)Cc1cc(C)cc(c1O)CN(CC(=O)O)Cc1cccc(n1)CN(CC(=O)O)C2] |
| I-8 | 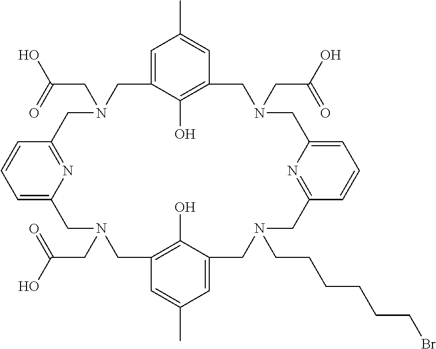 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCCBr)C2] |
| I-9 | 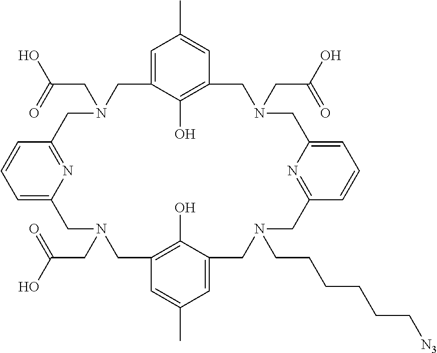 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCCN=[N+]=[N-])C2] |
| I-10 | 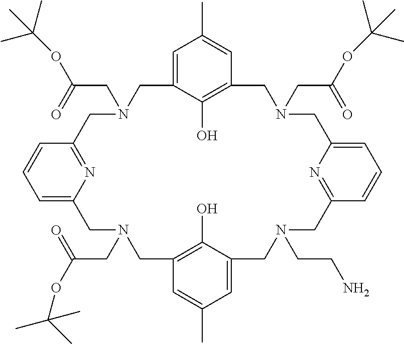 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCN)C2] |
| I-11 | 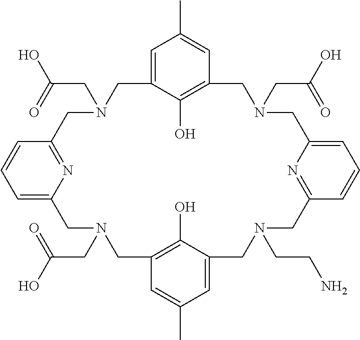 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCN)C2] |
| I-12 | 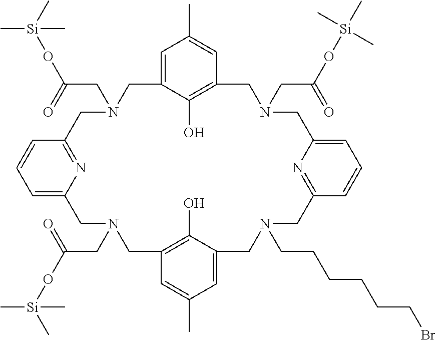 [SMILES: Cc1cc2c(O)c(c1)CN(CC(=O)O[Si](C)(C)C)Cc1cccc(n1)CN(CC(=O)O[Si](C)(C)C)Cc1cc(C)cc(c1O)CN(CC(=O)O[Si](C)(C)C)Cc1cccc(n1)CN(CCCCCCBr)C2] |
| I-13 | 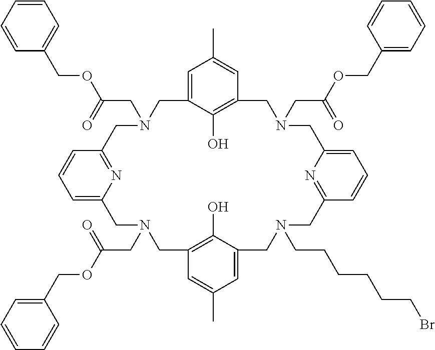 [SMILES: Cc1cc2c(O)c(c1)CN(CC(=O)OCc1ccccc1)Cc1cccc(n1)CN(CC(=O)OCc1ccccc1)Cc1cc(C)cc(c1O)CN(CC(=O)OCc1ccccc1)Cc1cccc(n1)CN(CCCCCCBr)C2] |
| I-14 | 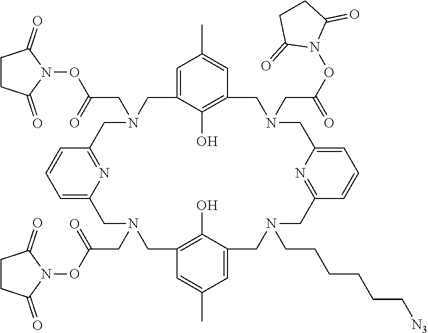 [SMILES: Cc1cc2c(O)c(c1)CN(CC(=O)ON1C(=O)CCC1=O)Cc1cccc(n1)CN(CC(=O)ON1C(=O)CCC1=O)Cc1cc(C)cc(c1O)CN(CC(=O)ON1C(=O)CCC1=O)Cc1cccc(n1)CN(CCCCCCN=[N+]=[N-])C2] |
| I-15 | 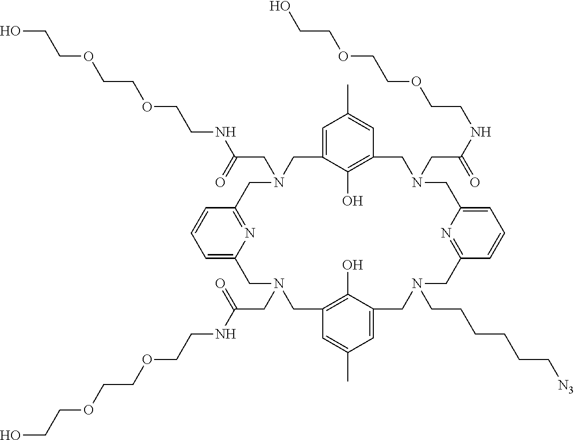 [SMILES: Cc1cc2c(O)c(c1)CN(CC(=O)NCCOCCOCCO)Cc1cccc(n1)CN(CC(=O)NCCOCCOCCO)Cc1cc(C)cc(c1O)CN(CC(=O)NCCOCCOCCO)Cc1cccc(n1)CN(CCCCCCN=[N+]=[N-])C2] |
| I-16 | 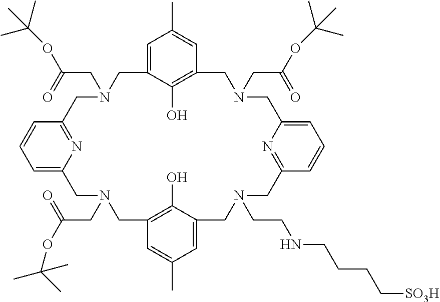 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCNCCCCS(=O)(=O)O)C2] |
| I-17 | 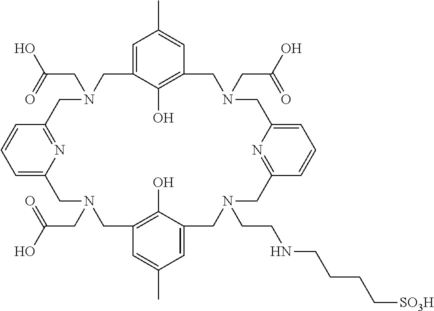 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCNCCCCS(=O)(=O)O)C2] |
| I-18 | 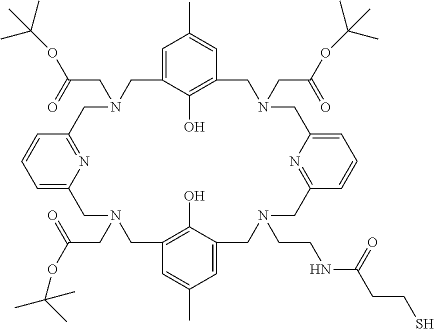 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCNC(=O)CCS)C2] |
| I-19 | 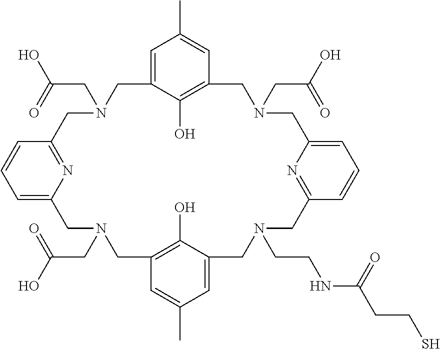 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCNC(=O)CCS)C2] |
| I-20 | 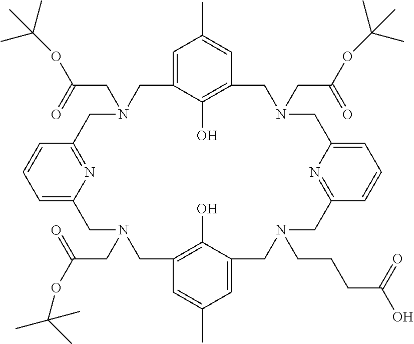 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCC(=O)O)C2] |
| I-21 | 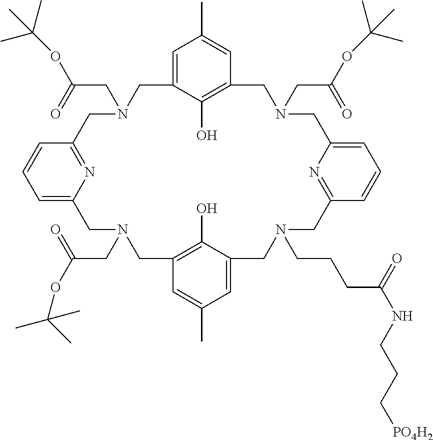 |
| I-22 | 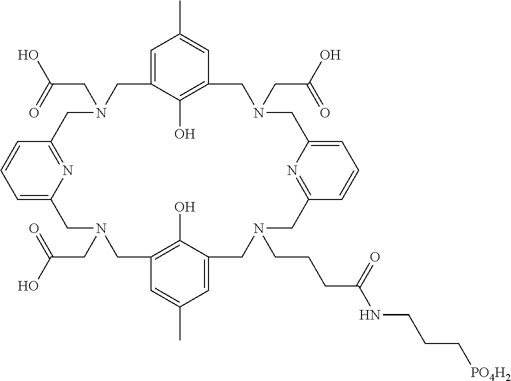 |
| I-23 | 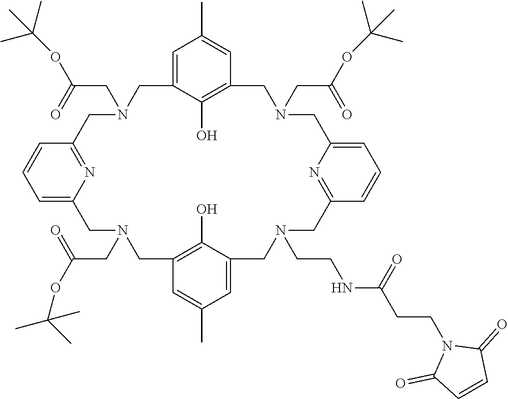 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCNC(=O)CCN1C(=O)C=CC1=O)C2] |
| I-24 | 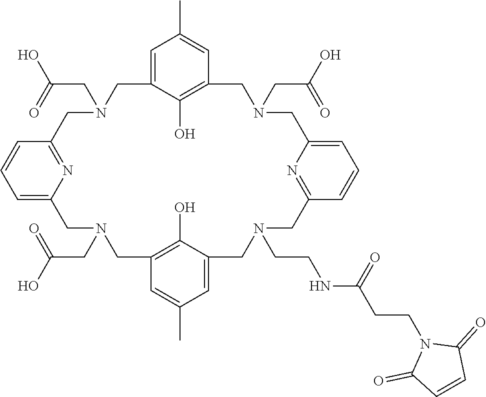 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCNC(=O)CCN1C(=O)C=CC1=O)C2] |
| I-25 | 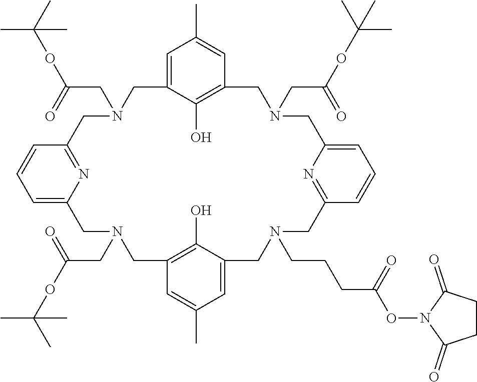 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCC(=O)ON1C(=O)CCC1=O)C2] |
| I-26 | 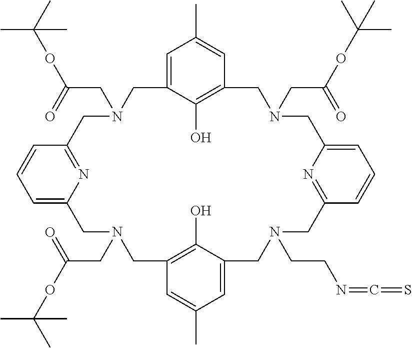 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCN=C=S)C2] |
| I-27 | 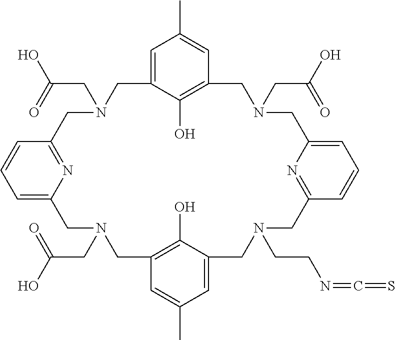 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCN=C=S)C2] |
| I-28 | 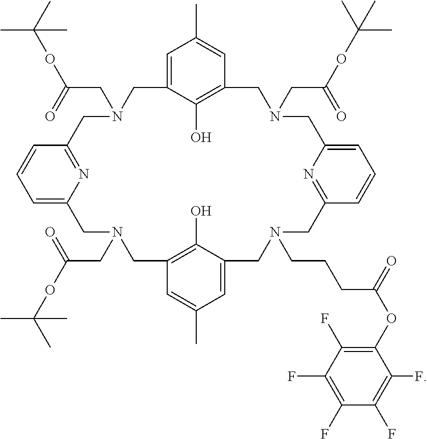 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCC(=O)Oc1c(F)c(F)c(F)c(F)c1F)C2] |

The term “halogen” refers to fluorine, chlorine, bromine, or iodine.

The term “C₁-C₁₂ alkyl” refers to a linear or branched saturated hydrocarbon group having 1 to 12 carbon atoms on the chain, including, without limitation, methyl, ethyl, propyl, isopropyl, butyl, isobutyl, sec-butyl, tert-butyl, etc.

The term “C₁-C₆ alkoxy” refers to a group formed by attaching an oxygen atom to the end of a C₁-C₆ alkyl, such as methoxy, ethoxy, n-propoxy, sec-butoxy, tert-butoxy, n-hexoxy, etc.

The term “C₁-C₁₂ silyl” is a structure of RₐR_{b}R_{c}Si—, wherein at least one of Rₐ, R_{b} and R_{c} is a C₁-C₁₂ alkyl group and the remainder is hydrogen, such as trimethylsilyl or triethylsilyl.

The term “oxygen-containing long-chain polymer group or derivative groups thereof” refers to a polymer group or derivative groups thereof containing oxygen, such as a polyethylene glycol group, a polypropylene glycol group, a polyethylene glycol derivative group of —X—CH₂CH₂(OCH₂CH₂)ₙ—OH, where X is NR (R is H or C₁-C₄ alkyl), O, S, etc.

The term “C₃-C₁₆ cycloalkyl” refers to a fully saturated monovalent cyclic hydrocarbon compound group containing 3 to 16 ring carbon atoms, with specific examples including cyclopropyl, cyclobutyl, cyclopentyl, and cyclohexyl. The meaning of C3-C6 cycloalkyl follows the same pattern.

The term “3-16 membered heterocyclic group” refers to a 3- to 16-membered non-aromatic cyclic group containing 1 to 4 heteroatoms selected from nitrogen, oxygen, sulfur, S(═O), and S(═O)₂ on the ring. Specific examples include oxetane, tetrahydroimidazole, and tetrahydrofuran.

The term “C₆-C₁₆ aryl” refers to an aryl group having 6 to 16 cyclic carbon atoms, and includes, without limitation, phenyl, naphthyl, etc.

The term “5- to 16-membered heteroaryl” refers to an aromatic group with 5 to 16 ring atoms containing 1 to 4 heteroatoms selected from nitrogen, oxygen, sulfur, S(═O), and S(═O)₂ on the ring. Specific examples thereof include pyridine, pyridazine, pyrimidine, and pyrrole, etc.

A “salt” refers to the corresponding salt formed by a molecule with a corresponding organic or inorganic acid, or a corresponding organic or inorganic base, such as hydrochloride, formate, trifluoroacetate, succinate, methanesulfonate salt of the compound, etc.

“Hydrate” refers to a compound that contains water.

The invention also includes any of the novel intermediates disclosed herein.

In a second aspect, the invention provides use of the compound of Formula I, or a precursor compound, isotopic compound, salt or hydrate thereof, as a macrocyclic chelating agent capable of site-specific modification in preparation of a nuclide probe.

In a third aspect, the invention provides a macrocyclic chelating agent conjugate formed by conjugating the compound of Formula I, or a precursor compound, isotopic compound, salt or hydrate thereof, to a target molecule.

In a preferred embodiment, the target molecule is any one selected from antibodies, peptides, saccharides, nucleic acids, small molecule drugs, optical dyes, organic nanomaterials, or fragments thereof or derivatives thereof.

Preferably, the antibody is selected from molecules containing a Fv fragment, and further selected from monoclonal antibodies, bispecific antibodies, and single-chain Fv fragments.

Preferably, the polypeptide is any one selected from octreotide, c(RGDyk), c(RGDfk), c(RADyk), folic acid, PSMA-617, and PSMA-11.

Preferably, the saccharide is any one selected from glucose, galactose, fructose, arabinose, rhamnose, ribose, mannose, lactose, maltose, amygdalin, and cyclodextrin.

Preferably, the nucleic acid is any one selected from deoxyribonucleic acid (DNA), ribonucleic acid (RNA), aptamers, and DNA/RNA hybrids.

Preferably, the small molecule drug is any one selected from afatinib, axitinib, bleomycin, camptothecin, capecitabine, dabrafenib, dasatinib, docetaxel, doxorubicin, and paclitaxel.

Preferably, the optical dye is any one selected from CH-1055, CH-4T, FD-1080, ICG, BODIPY, Rhodamine, IRdye800CW, and IR-1061.

Preferably, the organic nanomaterial is a liposome.

In a preferred embodiment, the macrocyclic chelating agent conjugate is selected from the following structures:

| No. | Chemical Structure |
|---|---|
| II-1 | 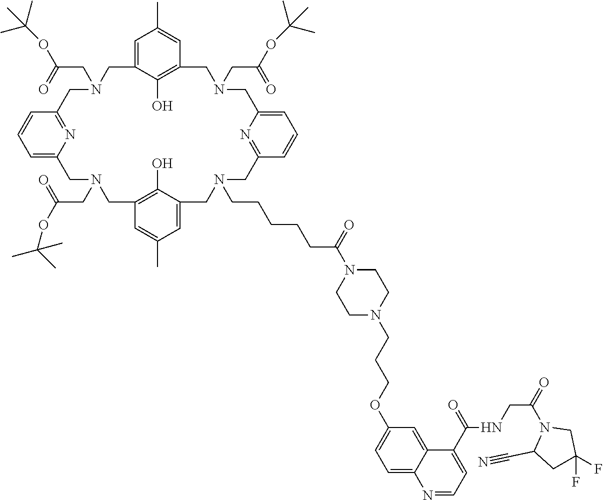 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCC(=O)N1CCN(CCCOc3ccc4nccc(C(=O)NCC(=O)N5CC(F)(F)CC5C#N)c4c3)CC1)C2] |
| II-2 | 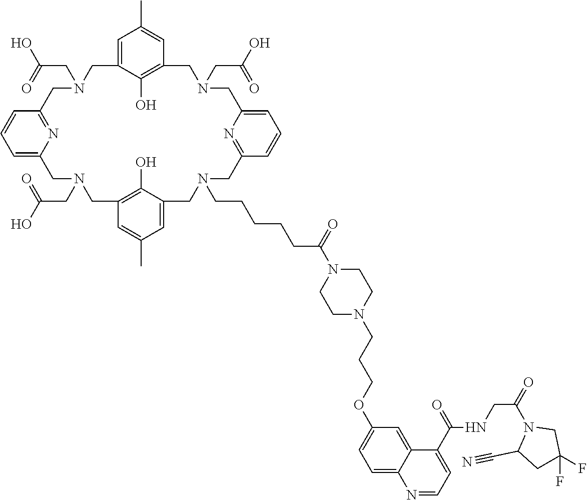 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCC(=O)N1CCN(CCCOc3ccc4nccc(C(=O)NCC(=O)N5CC(F)(F)CC5C#N)c4c3)CC1)C2] |
| II-3 | 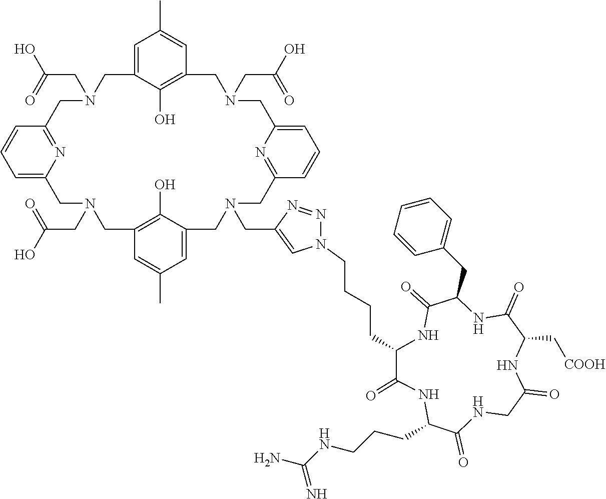 [SMILES: Cc1cc2c(O)c(c1)CN(CC(=O)O)Cc1cccc(n1)CN(Cc1cn(CCCC[C@@H]3NC(=O)[C@@H](Cc4ccccc4)NC(=O)[C@H](CC(=O)O)NC(=O)CNC(=O)[C@H](CCCNC(=N)N)NC3=O)nn1)Cc1cc(C)cc(c1O)CN(CC(=O)O)Cc1cccc(n1)CN(CC(=O)O)C2] |
| II-4 | 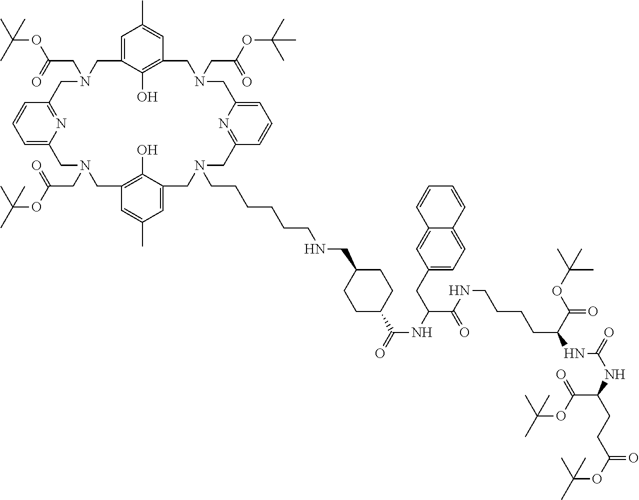 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCCNC[C@H]1CC[C@H](C(=O)NC(Cc3ccc4ccccc4c3)C(=O)NCCCC[C@H](NC(=O)N[C@@H](CCC(=O)OC(C)(C)C)C(=O)OC(C)(C)C)C(=O)OC(C)(C)C)CC1)C2] |
| II-5 | 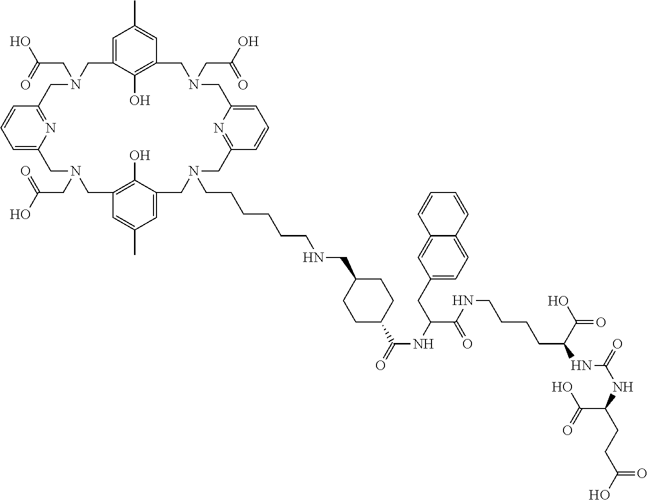 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCCNC[C@H]1CC[C@H](C(=O)NC(Cc3ccc4ccccc4c3)C(=O)NCCCC[C@H](NC(=O)N[C@@H](CCC(=O)O)C(=O)O)C(=O)O)CC1)C2] |
| II-6 | 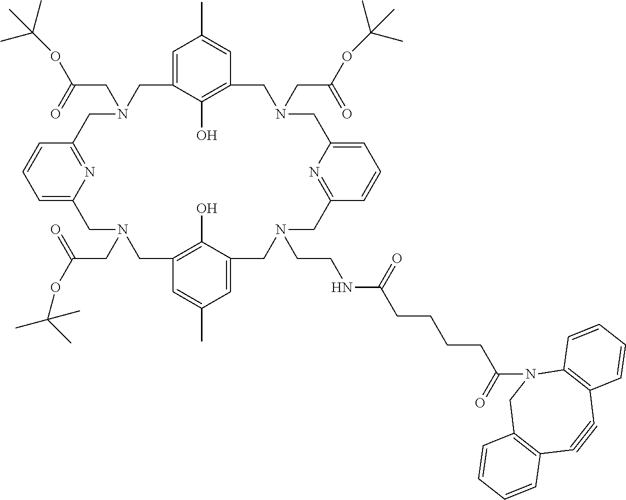 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCNC(=O)CCCCC(=O)N1Cc3ccccc3C#Cc3ccccc31)C2] |
| II-7 | 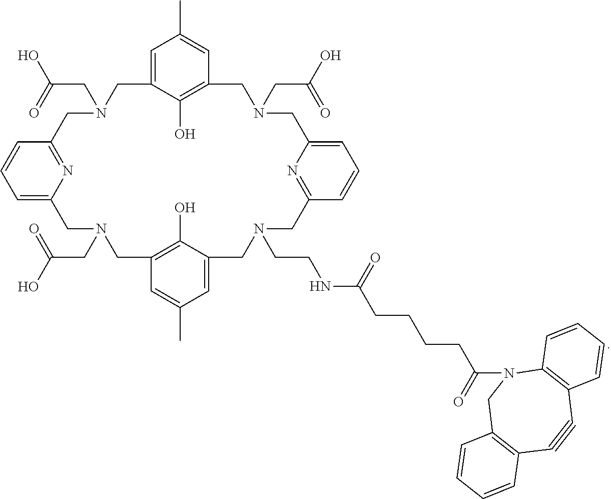 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCNC(=O)CCCCC(=O)N1Cc3ccccc3C#Cc3ccccc31)C2] |

In a fourth aspect, the invention provides a nuclide probe, which is the macrocyclic chelating agent conjugate labeled with a nuclide.

In a preferred embodiment, the nuclide is a radioactive nuclide or a non-radioactive nuclide.

Preferably, the radioactive nuclide is any one selected from ⁹⁰Y, ¹³¹I, ¹⁵³Sm, ¹⁷⁷Lu, ¹⁸⁶Re, ¹⁸⁸Re, ²¹¹At, ²¹²Pb, ²²³Ra, ²²⁵Ac, Al[¹⁸F], ⁵¹Mn, ^{52m}Mn, ^{52g}Mn, ⁶⁴Cu, ⁶⁷Cu, ⁶⁷Ga, ⁶⁸Ga, ⁸⁶Y, ⁸⁹Zr, ^{99m}Tc, ¹¹¹In, ¹²³I, ¹²⁴I, ¹²⁵I, ¹⁶⁶Ho, ²¹³Bi, ²²⁷Th, etc.

Preferably, the non-radioactive nuclide is any one selected from Mn, Si, Pt, Nd, Gd, Yb, Er, Cu, Y, Fe, Tm, etc.

In a fifth aspect, the invention provides use of the nuclide probe in molecular imaging and radionuclide therapy.

The molecular imaging includes, but is not limited to, PET imaging, SPECT imaging, optical imaging (CT imaging), MRI imaging, multimodal imaging formed by mutual combinations of these imaging techniques, and radionuclide therapy.

In a sixth aspect, the invention provides use of the nuclide probe in preparation of a molecular imaging diagnostic agent or tumor therapeutic drug.

The molecular imaging diagnostic agent can be a PET and/or SPECT molecular imaging diagnostic agent; and the tumor therapeutic drug can be a radionuclide therapy drug.

### Beneficial Effects:

The macrocyclic chelating agent of the present invention can site-specifically modify various target molecules including antibodies, peptides, saccharides, nucleic acids, small molecule drugs, optical dyes, organic nanomaterials or fragments or derivatives thereof, at the R₄ site, with improved accurate and efficient reaction. At the same time, it can broadly chelate various nuclides, showing great promise in the diagnosis and treatment of nuclear imaging.

The present invention has been described in detail above; however, the above embodiments are merely illustrative in nature and are not intended to limit the invention. Furthermore, the present invention is not limited to any theory described in the foregoing prior art or in the summary, or in the following examples.

### BRIEF DESCRIPTION OF DRAWINGS

FIG. 1 shows ¹H NMR spectrum of compound I-5.

FIG. 2 shows labeling rates of compound I-5 with different masses labeled with ⁶⁸Ga at 25° C. and 37° C.

FIG. 3 shows PET/CT images of compound II-2 labeled with ⁶⁸Ga in U87MG tumor mice.

FIG. 4 shows high performance liquid chromatogram of the reaction solution for the synthesis of compound Dar-PSMA (Dar-1).

FIG. 5 shows high-performance liquid chromatogram of the reaction solution for the synthesis of compound I-1.

FIG. 6 shows high-performance liquid chromatogram of the reaction solution for the synthesis of compound I-2.

FIG. 7 shows high-performance liquid chromatogram of the reaction solution for the synthesis of compound 6a.

### DETAILED DESCRIPTION OF THE INVENTION

The present invention will be further described below with reference to the examples. It should be noted that the following examples are provided for illustrative purposes only and do not constitute a limitation on the scope of protection of the present invention.

Unless otherwise specified, the raw materials, reagents, and methods used in the examples are all conventional raw materials, reagents, and methods in the art.

Raw materials: Unless otherwise specified, all compounds used were purchased commercially.

### EXAMPLE

### Example 1: Synthesis of Compound I-1

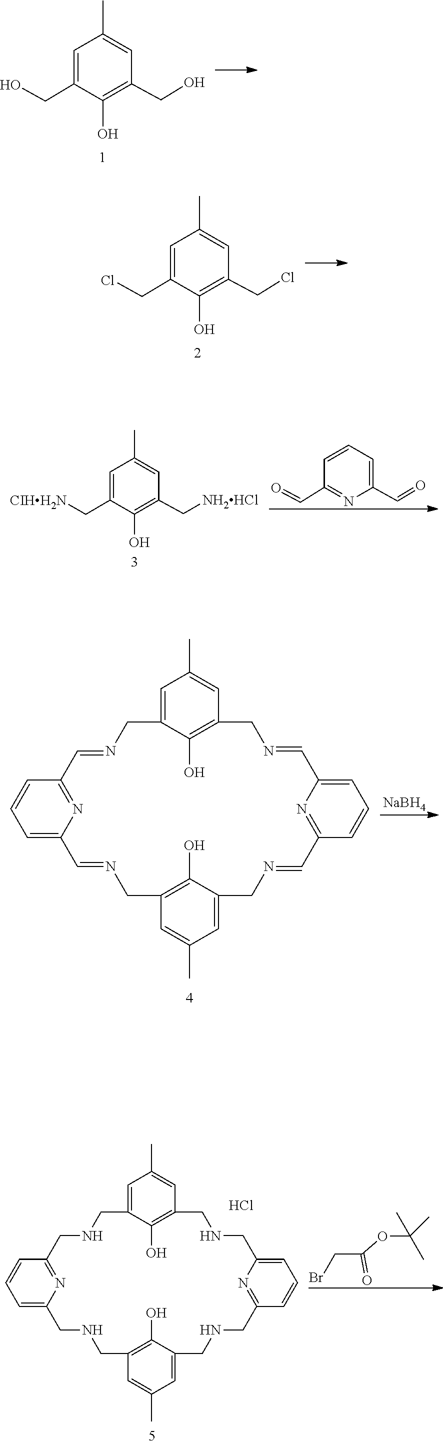

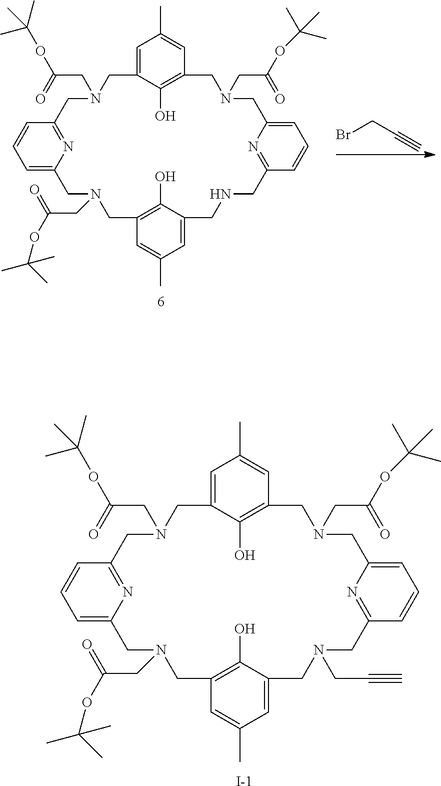

Compound 1 (10 g, 59.4 mmol) was added in a reaction flask with 12 mL of dichloromethane, and then 60 mL of concentrated hydrochloric acid was added. The mixture was stirred at 25° C. for 24 h. After TLC showed complete reaction, the solvent was removed by rotary evaporation to obtain 12 g of crude compound 2, with a crude yield of 100%.

27 g of hexamethylenetetramine (urotropin) was dissolved in 240 mL of methanol, and the crude compound 2 (12 g, 58.5 mmol) was quickly added thereto. The mixture was stirred at room temperature for 4 h, and filtered to collect the precipitate. The precipitate was added to a mixed solvent of 100 mL of ethanol and 38 mL of 32% concentrated hydrochloric acid, heated to 80° C. and stirred for 8 h. The reaction solvent was removed by rotary evaporation, and the residue was added with 150 mL of ethanol, stirred for 2 h and filtered, and the filtrate was collected. 150 mL of n-hexane was added to the filtrate, stirred for 2 h, and filtered to collect the solid product. 3 g of compound 3 was obtained with a yield of 25%.

Compound 3 (2.5 g, 12.3 mmol) was dissolved in 75 mL of methanol, and N,N-diisopropylethylamine (4.5 mL, 25.9 mmol) and 2,6-pyridinedicarboxaldehyde (1.66 g, 12.3 mmol) were added. The mixture was heated to 55° C. and reacted for 2 hours, followed by reacted at room temperature overnight. After the reaction was complete, the precipitate was collected by filtration and dried under vacuum to obtain 2.75 g of crude compound 4.

Compound 4 (2.75 g, 5.18 mmol) was dispersed in 500 mL of methanol and heated to 45° C. NaBH₄ (8 g, 211 mmol) was added, and the reaction was carried out at this temperature for 3 hours. The solvent was removed by rotary evaporation, and the mixture was extracted with a mixture of dichloromethane and water. The organic phase was washed with saturated brine, dried over anhydrous sodium sulfate, and the solvent was removed by rotary evaporation. 25 mL of 36% hydrochloric acid-methanol solution was added, and the mixture was stirred overnight at 0° C. The precipitated solid was collected by filtration and dried under vacuum to obtain 1.01 g of compound 5. The overall yield of the two steps was 30%.

Compound 5 (500 mg, 0.93 mmol) was dispersed in 40 mL of acetonitrile, and N,N-diisopropylethylamine (932 μL, 5.35 mmol) and tert-butyl bromoacetate (478 mg, 2.45 mmol) were added. The mixture was stirred and reacted at room temperature for 30 min. After the reaction was complete, the solvent was removed by rotary evaporation, and the residue was purified by silica gel column chromatography to give 160 mg of compound 6, with a yield of 19%.

Compound 6 (30 mg, 0.034 mmol) was dispersed in 2 mL of acetonitrile, and N,N-diisopropylethylamine (16 μL, 0.093 mmol) and 3-bromopropyne (6 mg, 0.05 mmol) were added. The mixture was stirred and reacted at room temperature for 30 min. After the reaction was complete, the solvent was removed by rotary evaporation, and the residue was purified by silica gel column chromatography to obtain 29 mg of compound I-1, with a yield of 93%. ESI-MS: Calc.: 918.53; Found: 919.6 [M+H].

### Example 2: Synthesis of Compounds I-2 and II-4

### 1. Synthesis of Compound I-2

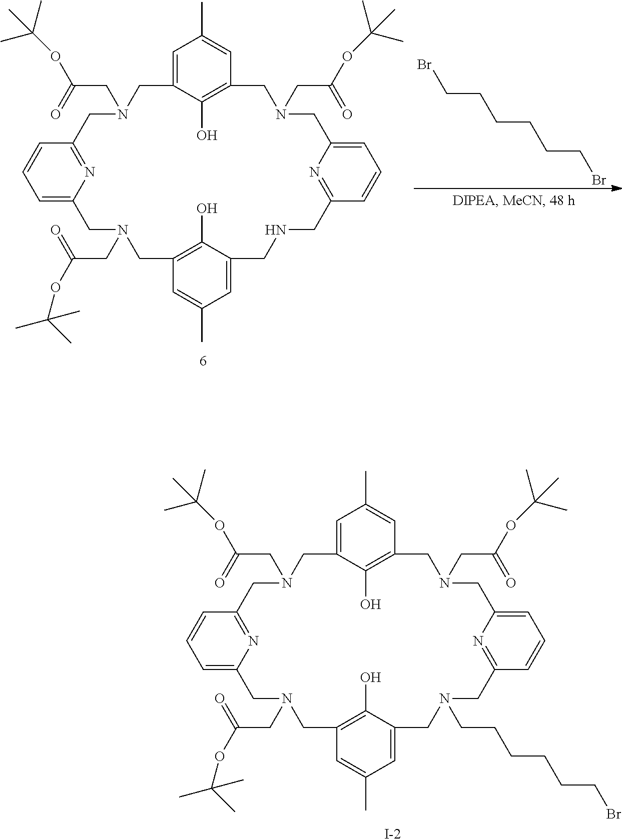

Compound 6 (100 mg, 0.011 mmol) was dispersed in 2 mL of acetonitrile, and N,N-diisopropylethylamine (10 μL, 0.058 mmol) and 1,6-dibromohexane (4 mg, 0.016 mmol) were added. The mixture was stirred and reacted at 60° C. for 48 hours. After the reaction was complete, the solvent was removed by rotary evaporation, and the residue was purified by semi-preparative high-performance liquid chromatography to obtain 80 mg of compound I-2, with a yield of 68%. ESI-MS: Calc.: 1042.51/1044.51; Found: 1043.6/1045.6 [M+H].

### 2. Synthesis of Macrocyclic Chelating Agent Conjugate II-4

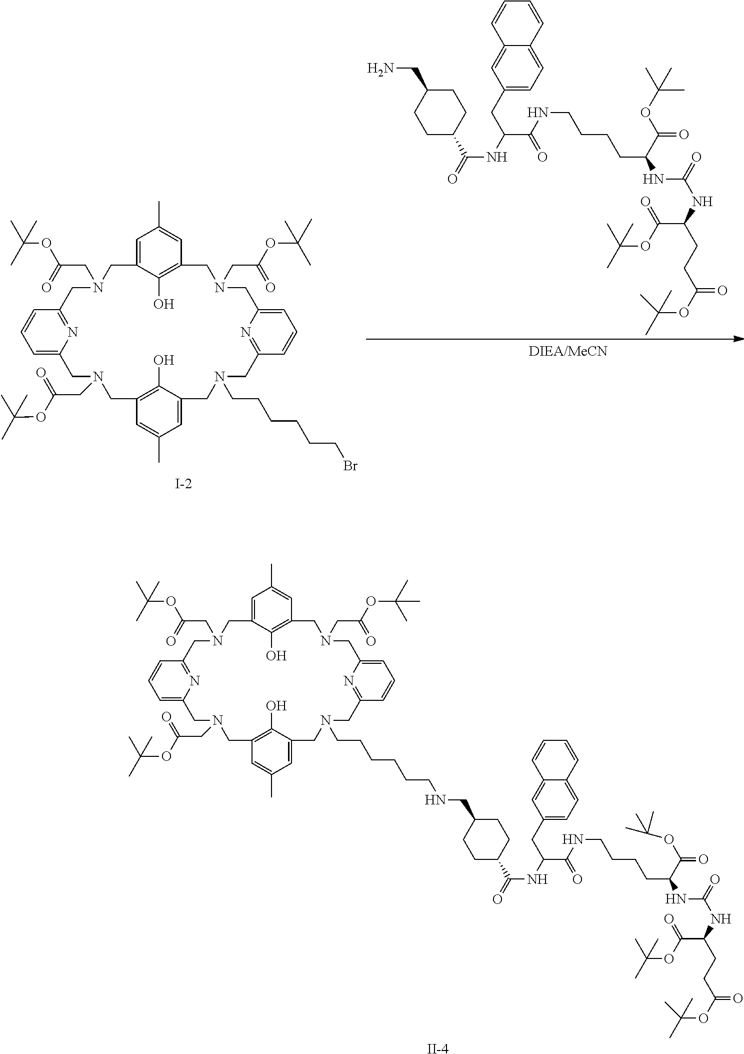

Compound I-2 (5 mg, 0.0048 mmol), a PSMA-617 derivative (CAS: 1703768-74-4, 5.9 mg, 0.0072 mmol), and N,N-diisopropylethylamine (4.2 μL, 0.024 mmol) were added to acetonitrile and stirred overnight at 60° C. The mixture was purified by semi-preparative high-performance liquid chromatography (HPLC) to obtain 3.9 mg of compound II-4, with a yield of 46%. ESI-MS: Calc.: 1786.10; Found: 1787.3 [M+H].

### Example 3: Synthesis of Compounds I-3 and II-1

### 1. Synthesis of Compound I-3

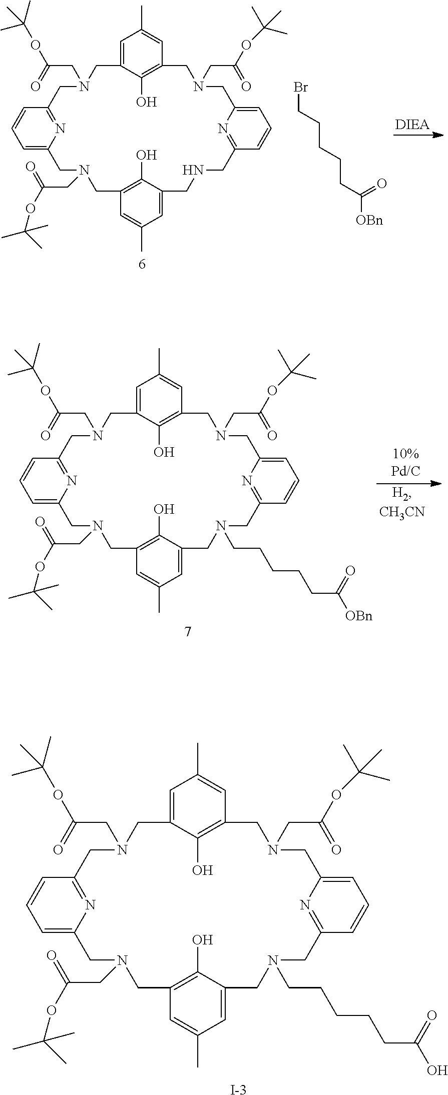

Compound 6 (10 mg, 0.011 mmol) was dispersed in 2 mL of acetonitrile, and N,N-diisopropylethylamine (10 μL, 0.058 mmol) and benzyl 6-bromohexanoate (3.8 mg, 0.013 mmol) were added. The mixture was stirred and reacted at 60° C. for 16 hours. After the reaction was complete, the solvent was removed by rotary evaporation, and the product was used directly in the next reaction without purification.

The crude product obtained in the previous step was dissolved in 5 mL of acetonitrile, and 1 mg of 10% Pd/C was added. The reaction was carried out under hydrogen atmosphere for 6 hours. The reaction was stopped, and 5.2 mg of compound I-3 was obtained by semi-preparative high-performance liquid chromatography. The overall yield of the two steps was 46%. ESI-MS: Calc.: 994.58; Found: 995.8 [M+H].

### 2. Synthesis of Macrocyclic Chelating Agent Conjugate II-1

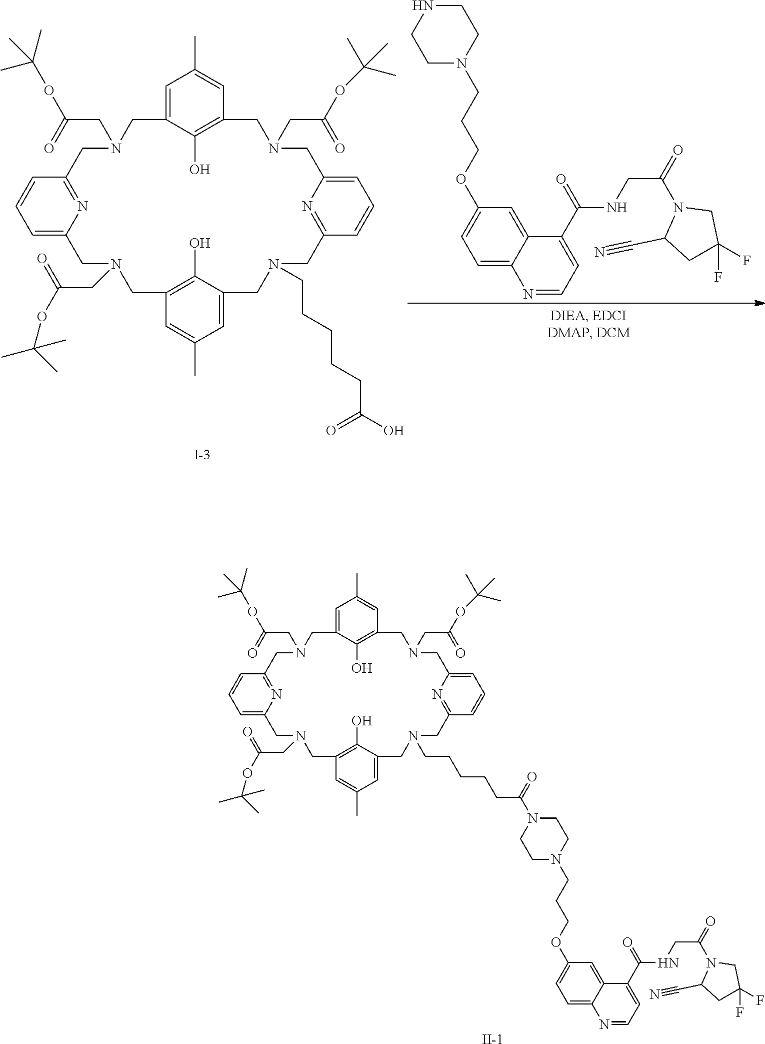

Compound I-3 (6.0 mg, 0.006 mmol) was dispersed in 2 mL of dichloromethane. N,N-diisopropylethylamine (5.3 μL, 0.031 mmol), an FAPI analog (CAS No.: 2471983-20-5, 3.5 mg, 0.007 mmol), 1-(3-dimethylaminopropyl)-3-ethylcarbodiimide hydrochloride (2.3 mg, 0.012 mmol), and 4-dimethylaminopyridine (catalytic amount, 0.1 mg) were added. The mixture was stirred and reacted overnight at room temperature. After the reaction was complete, the solvent was removed by rotary evaporation, and the residue was purified by semi-preparative high-performance liquid chromatography to obtain 5.2 mg of compound II-1, with a yield of 59%. ESI-MS: Calc.: 1462.79; Found: 1463.8 [M+H].

### Example 4: Synthesis of Compound I-4

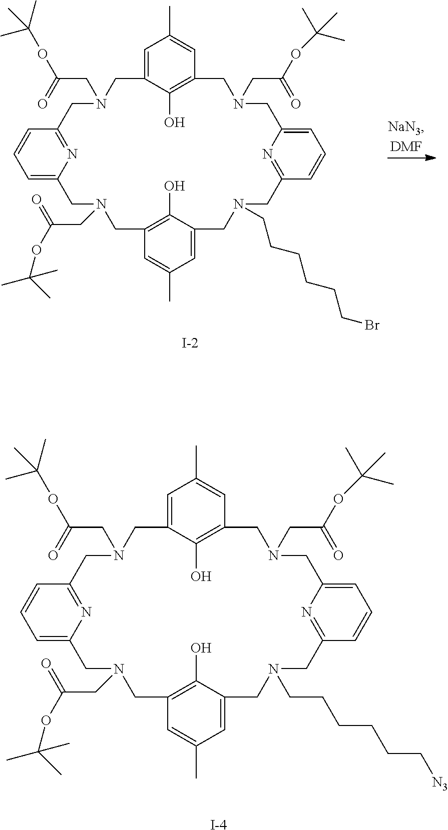

Compound I-2 (10 mg, 0.01 mmol) was added to 2 mL of N,N-dimethylformamide, followed by addition of sodium azide (6.5 mg, 0.1 mmol). The mixture was reacted overnight at 70° C. After cooling to room temperature, 5 mL of saturated ammonium chloride was added, and the mixture was extracted with dichloromethane. The organic phase was washed with saturated sodium chloride and dried over anhydrous sodium sulfate, and the solvent was removed by rotary evaporation. The residue was purified by semi-preparative high-performance liquid chromatography to obtain 8.1 mg of compound I-4, with a yield of 84%. ESI-MS: Calc.: 1005.61; Found: 1006.7 [M+H].

### Example 5: Synthesis of Compound I-5

### 1. Synthesis of Compound I-5

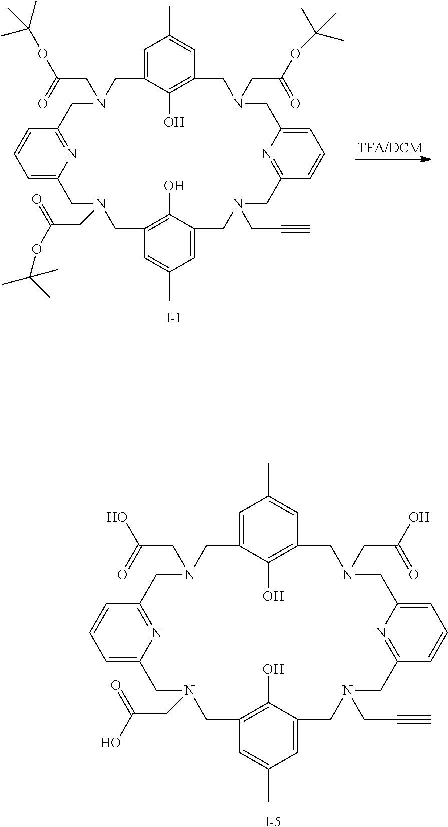

Compound I-1 (10 mg, 0.011 mmol) was added to a mixed solvent of 0.5 mL of dichloromethane and 0.5 mL of trifluoroacetic acid, stirred overnight at room temperature, and the solvent was removed by rotary evaporation. The residue was dissolved in 0.5 mL of dichloromethane, and then precipitated by adding 5 mL anhydrous diethyl ether. The precipitate was collected by centrifugation to give 6.7 mg of compound I-5, with a yield of 82%. ESI-MS: Calc.: 750.34; Found: 751.2 [M+H].

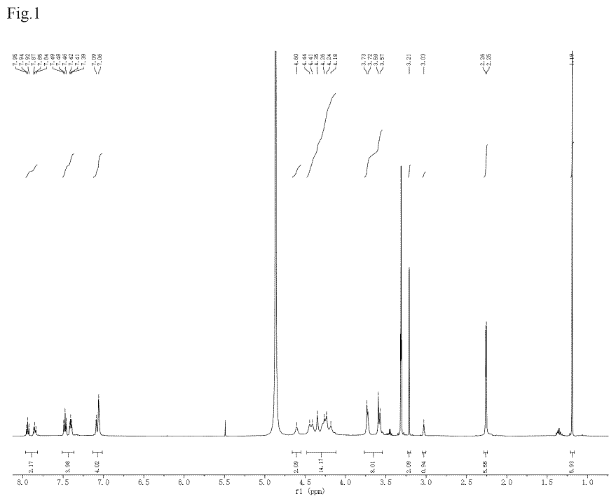

4 mg of compound I-5 was dissolved in deuterated methanol and analyzed on a Bruker 600 MHz instrument to obtain 1H NMR data. The results are shown in FIG. 1.

### 2. Synthesis of Macrocyclic Chelating Agent Conjugate II-3

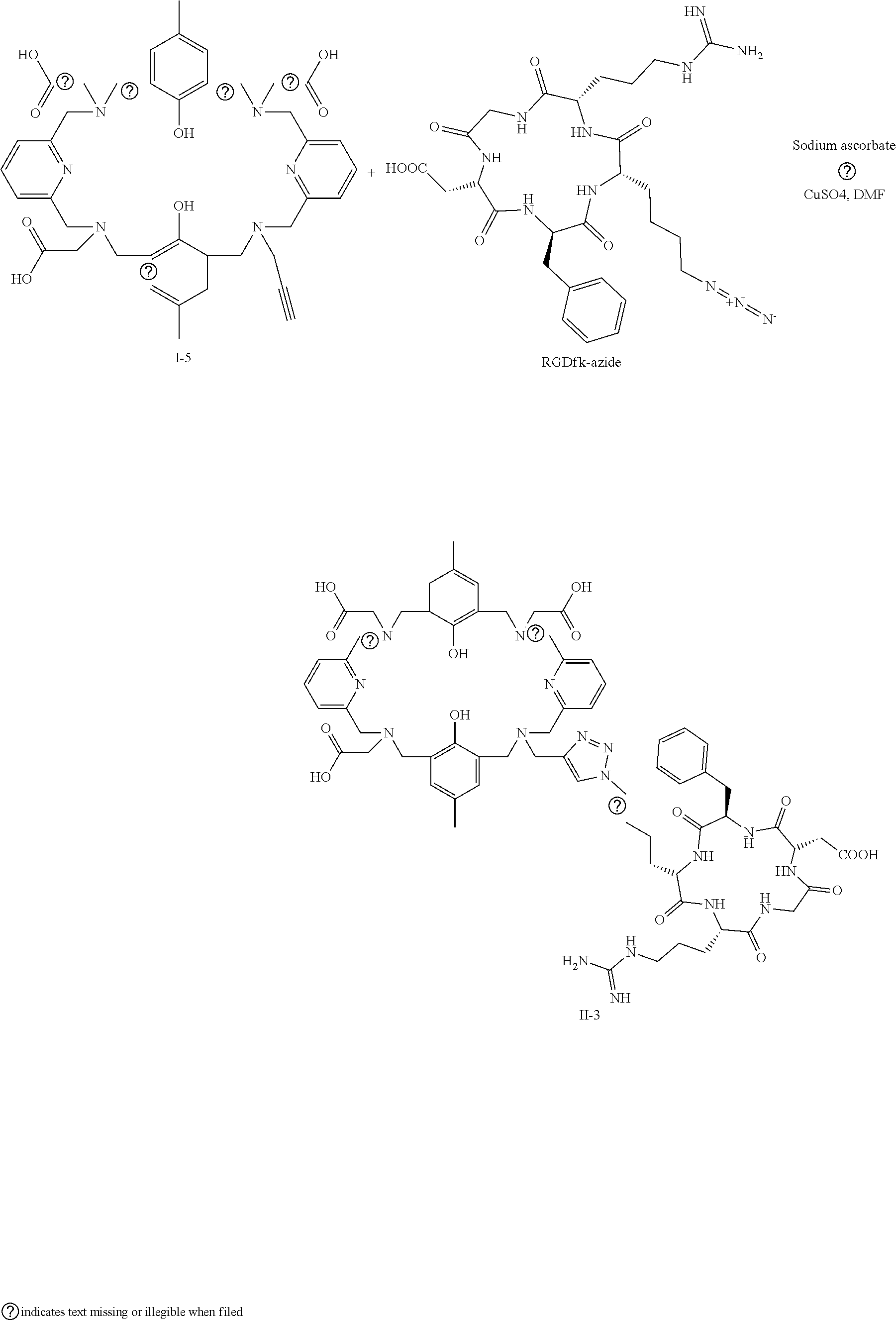

Compound I-5 (1 mg, 0.0013 mmol), sodium ascorbate (0.2 mg, 0.0007 mmol), copper sulfate (0.1 mg, 0.0004 mmol), and cyclic peptide cRGDfk-azide (1.2 mg, 0.002 mmol) were reacted in 2 mL of N,N-dimethylformamide overnight at 70° C. The mixture was purified by semi-preparative high-performance liquid chromatography to obtain 480 μg of compound II-3, with a yield of 26%. ESI-MS: Calc.: 1379.64; Found: 1380.8 [M+H].

### Example 6: Synthesis of Compounds I-6 and II-2

### 1. Synthesis of Compound I-6

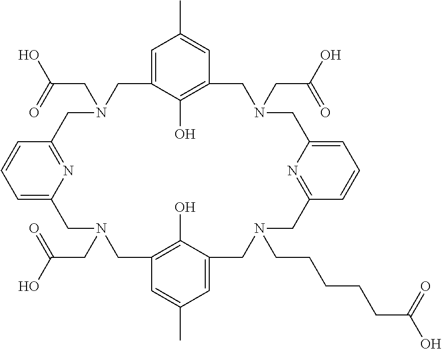 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCC(=O)O)C2]

The compound I-6 was synthesized the same as that of I-5, except that compound I-1 was replaced with I-3.

### 2. Synthesis of Macrocyclic Chelating Agent Conjugate II-2

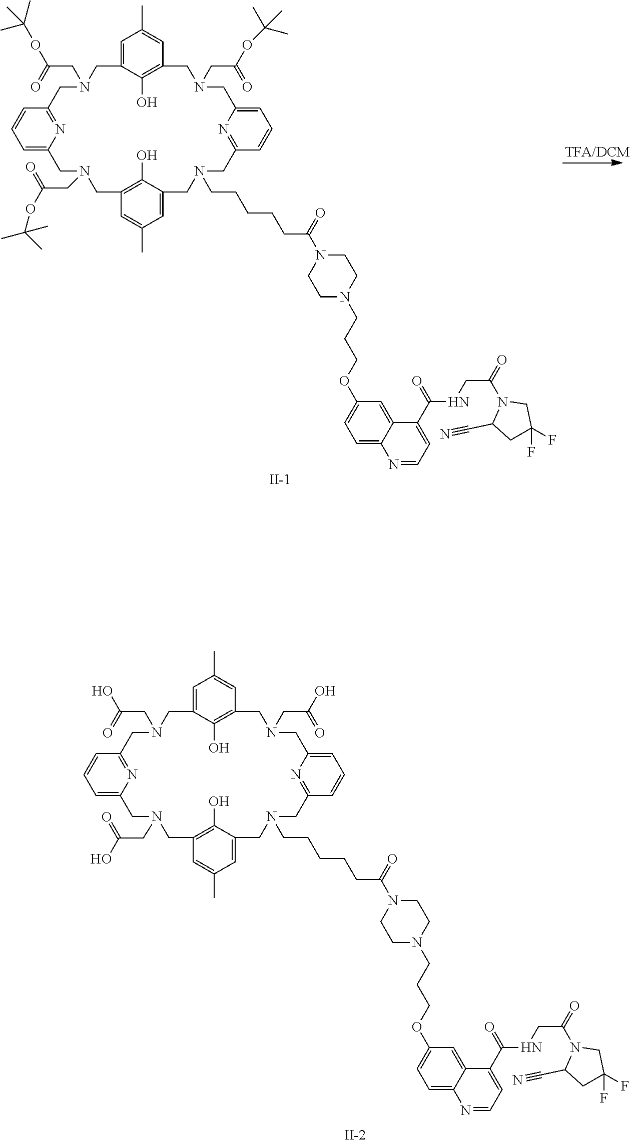 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCC(=O)N1CCN(CCCOc3ccc4nccc(C(=O)NCC(=O)N5CC(F)(F)CC5C#N)c4c3)CC1)C2 ; "II-1" ; Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCC(=O)N1CCN(CCCOc3ccc4nccc(C(=O)NCC(=O)N5CC(F)(F)CC5C#N)c4c3)CC1)C2 ; "II-2"]

Compound II-1 (1 mg, 0.0007 mmol) was added to a mixed solvent of 0.5 mL of dichloromethane and 0.5 mL of trifluoroacetic acid. The mixture was stirred overnight at room temperature, and the solvent was removed by rotary evaporation. The residue was purified by semi-preparative high-performance liquid chromatography to obtain 590 μg of compound II-2, with a yield of 67%. ESI-MS: Calc.: 1294.60; Found: 1295.5 [M+H].

### Example 7: Synthesis of Compound I-7

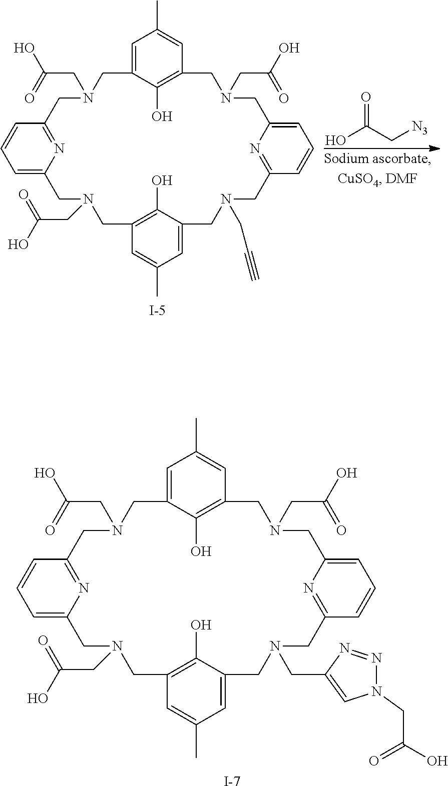

Compound I-5 (5 mg, 0.0067 mmol), sodium ascorbate (0.7 mg, 0.0033 mmol), copper sulfate (0.4 mg, 0.0017 mmol), and azidoacetic acid (1 mg, 0.01 mmol) were reacted in 2 mL of N,N-dimethylformamide overnight at 70° C. The mixture was purified by semi-preparative high-performance liquid chromatography to obtain 1.6 mg of compound I-7, with a yield of 28%. ESI-MS: Calc.: 851.36; Found: 852.4 [M+H].

### Example 8: Synthesis of Compound I-8

### 1. Synthesis of Compound I-8

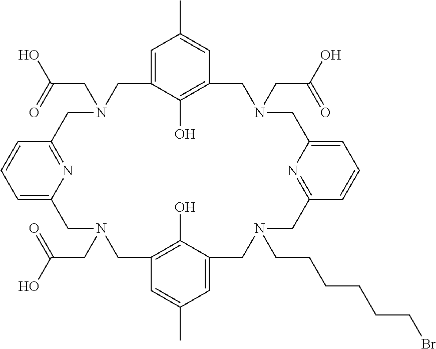 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCCBr)C2]

The compound I-8 was synthesized the same as that of I-5, except that compound I-1 was replaced with I-2.

### 2. Synthesis of Macrocyclic Chelating Agent Conjugate II-5

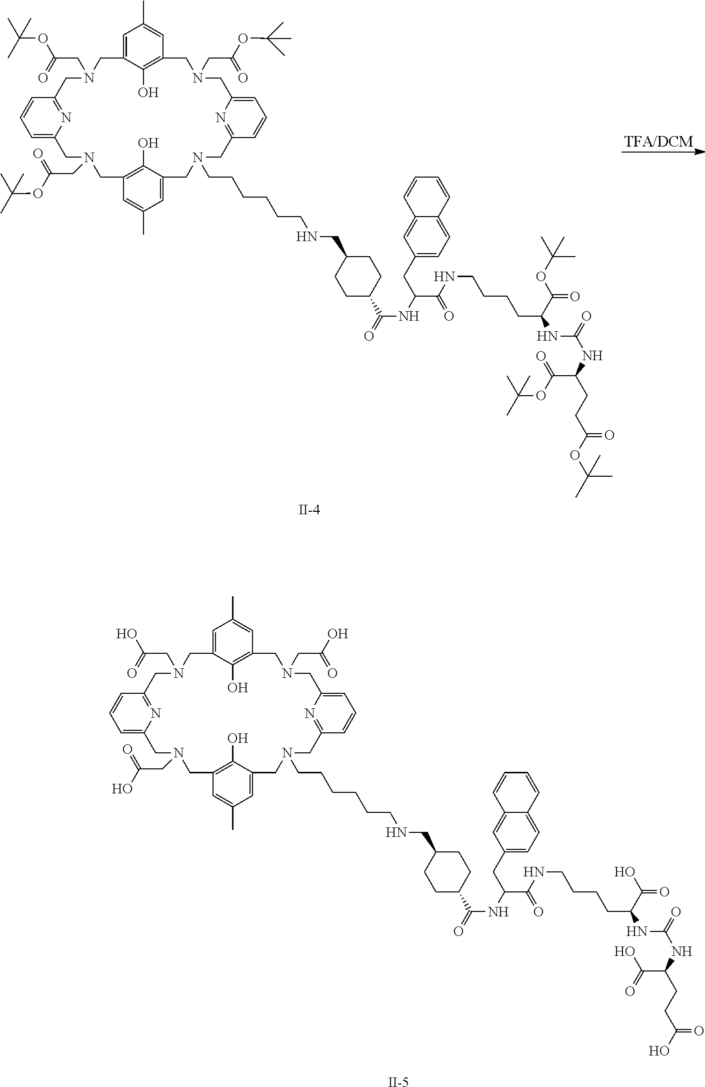 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCCNC[C@H]1CC[C@H](C(=O)NC(Cc3ccc4ccccc4c3)C(=O)NCCCC[C@H](NC(=O)N[C@@H](CCC(=O)OC(C)(C)C)C(=O)OC(C)(C)C)C(=O)OC(C)(C)C)CC1)C2 ; "II-4" ; Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCCNC[C@H]1CC[C@H](C(=O)NC(Cc3ccc4ccccc4c3)C(=O)NCCCC[C@H](NC(=O)N[C@@H](CCC(=O)O)C(=O)O)C(=O)O)CC1)C2 ; "II-5"]

Compound II-4 (2 mg, 0.0011 mmol) was added to a mixed solvent of 0.5 mL of dichloromethane and 0.5 mL of trifluoroacetic acid. The mixture was stirred overnight at room temperature, and the solvent was removed by rotary evaporation. The residue was dissolved in 0.5 mL of dichloromethane, and then precipitated by adding 5 mL of anhydrous diethyl ether. The precipitate was collected by centrifugation to obtain 1.2 mg of compound II-5, with a yield of 74%. ESI-MS: Calc.: 1449.72; Found: 1450.9 [M+H].

### Example 9: Synthesis of Compound I-9

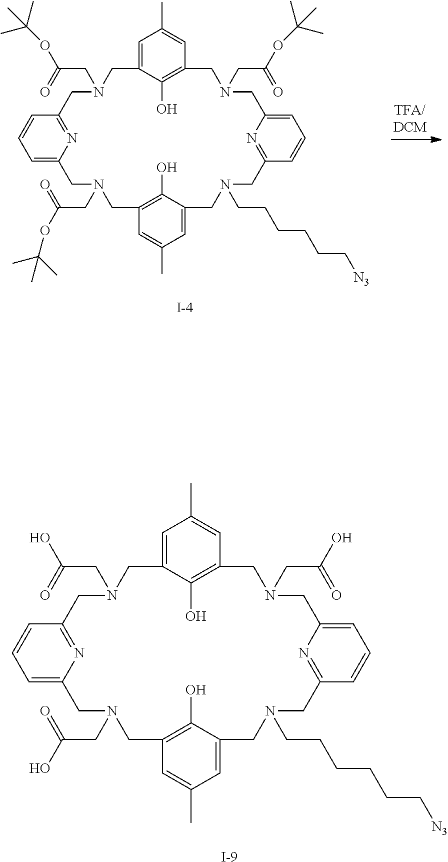

Compound I-4 (5 mg, 0.0054 mmol) was added to a mixed solvent of 1 mL of dichloromethane and 1 mL of trifluoroacetic acid, and stirred overnight at room temperature, and the solvent was removed by rotary evaporation. The residue was dissolved in 0.5 mL of dichloromethane, and precipitated by adding 5 mL of diethyl ether. The precipitate was dried to give 3 mg of compound I-9, with a yield of 72%. ESI-MS: Calc.: 837.42; Found: 838.7 [M+H].

### Example 10: Synthesis of Compounds I-10 and II-6

### 1. Synthesis of Compound I-10

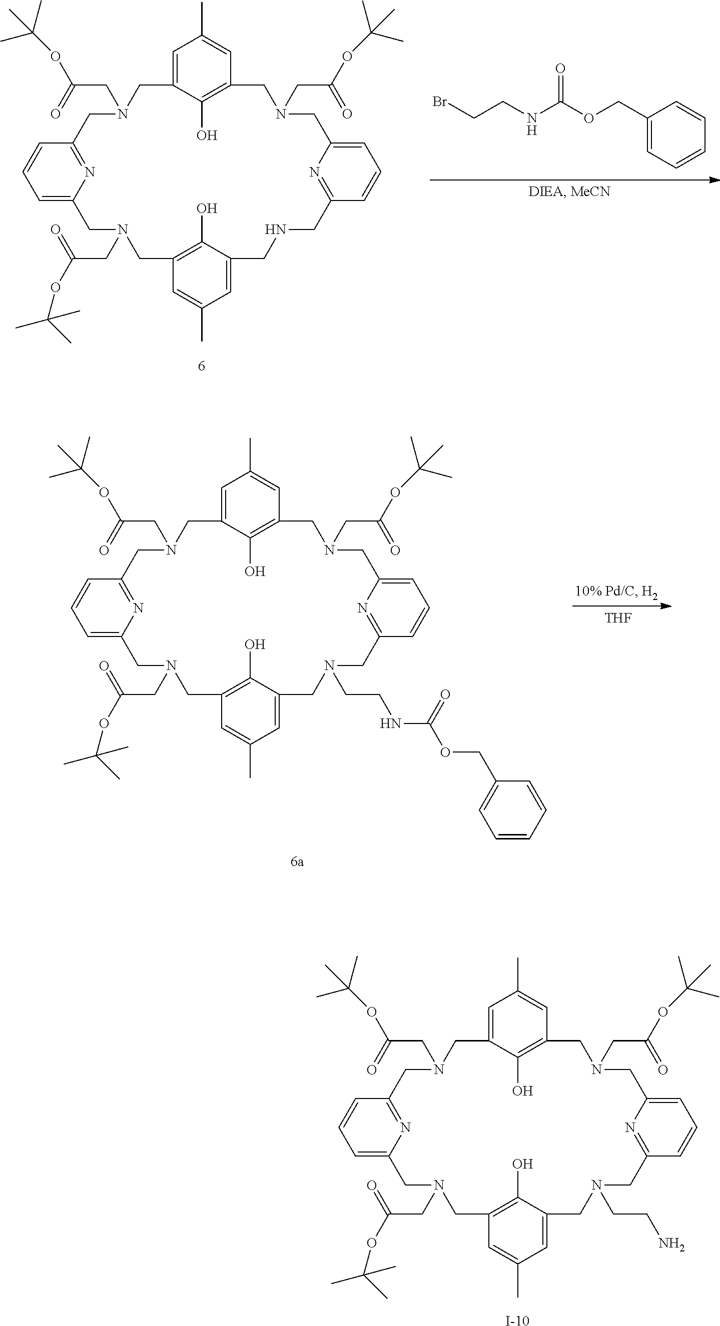

Compound 6 (50 mg, 0.057 mmol) was added to 10 mL of acetonitrile, followed by addition of N,N-diisopropylethylamine (50 μL, 0.284 mmol) and N-benzyloxycarbonyl-3-bromoethylamine (29.4 mg, 0.114 mmol). The mixture was stirred overnight at room temperature, and the solvent was removed by rotary evaporation. The residue was purified by silica gel column chromatography to obtain 57 mg of compound 6a.

Compound 6a (57 mg, 0.054 mmol) was dissolved in 10 mL of tetrahydrofuran, and 10 mg of 10% Pd/C was added. The reaction was carried out under hydrogen atmosphere for 12 h. The solvent was removed by rotary evaporation, and the residue was purified by silica gel column chromatography to obtain 40 mg of compound I-10. The overall yield of the two steps was 76%. ESI-MS: Calc.: 923.55; Found: 924.8 [M+H].

### 2. Synthesis of Macrocyclic Chelating Agent Conjugate II-6

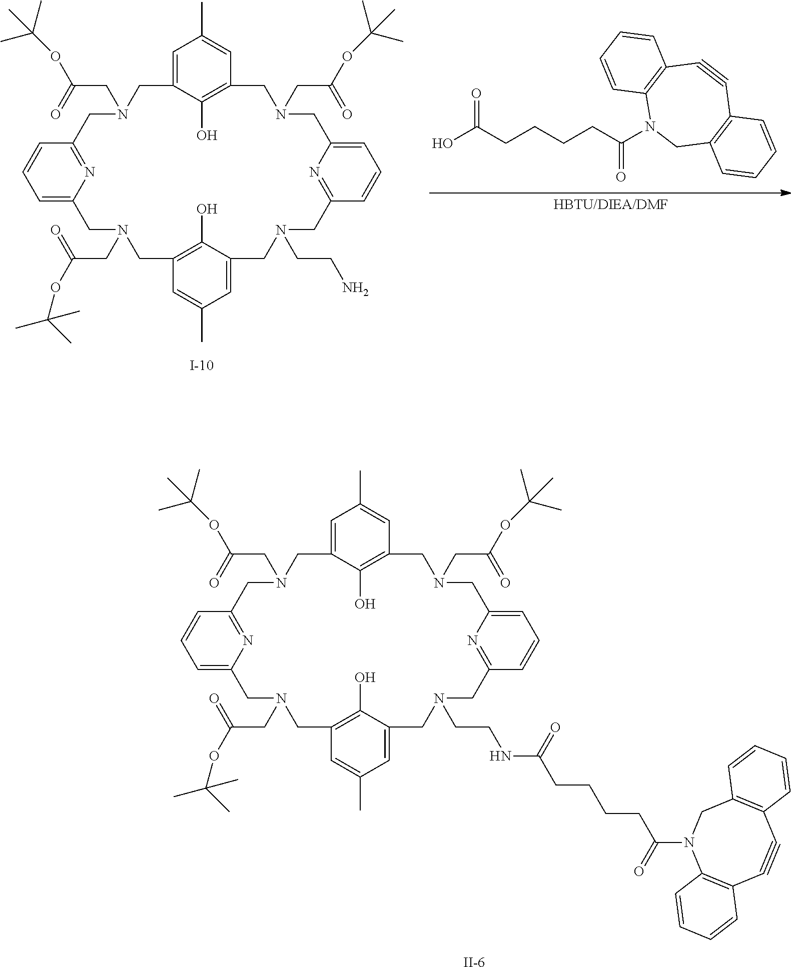

Compound I-10 (10 mg, 0.011 mmol), DBCO-C₆-acid (CAS: 1425485-72-8, 3.6 mg, 0.011 mmol), N,N,N′,N′-tetramethyl-O-(7-azabenzotriazol-1-yl)uronium hexafluorophosphate (208 mg, 0.55 mmol), and N,N-diisopropylethylamine (96 μL, 0.55 mmol) were added to 10 mL of N,N-dimethylformamide and reacted overnight at room temperature. The mixture was purified by semi-preparative high-performance liquid chromatography to obtain 7 mg of compound II-6, with a yield of 52%. ESI-MS: Calc.: 1238.68; Found: 1239.9 [M+H].

### Example 11: Synthesis of Compounds I-11 and II-7

### 1. Synthesis of Compound I-11

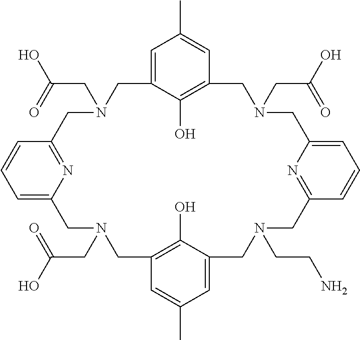 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCN)C2]

The compound I-11 was synthesized the same as that of I-5, except that compound I-1 was replaced with I-10.

### 2. Synthesis of Macrocyclic Chelating Agent Conjugate II-7

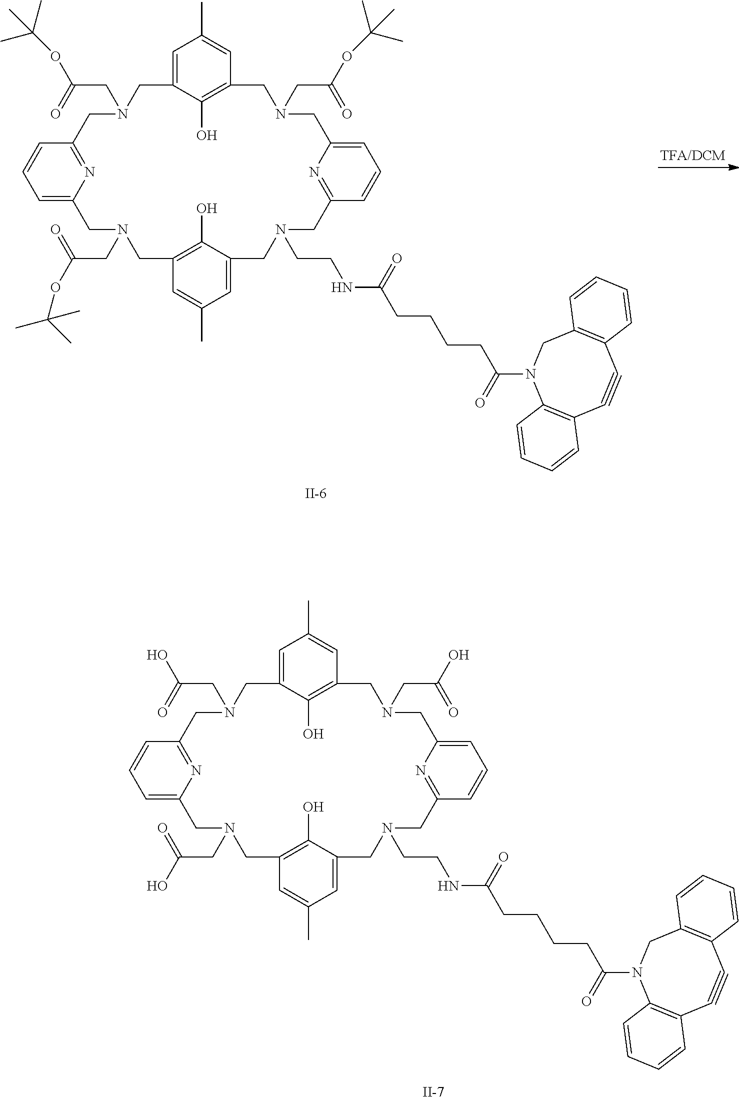 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCNC(=O)CCCCC(=O)N1Cc3ccccc3C#Cc3ccccc31)C2 ; "II-6" ; Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCNC(=O)CCCCC(=O)N1Cc3ccccc3C#Cc3ccccc31)C2 ; "II-7"]

Compound II-6 (2 mg, 0.0016 mmol) was added to a mixed solvent of 0.5 mL of dichloromethane and 0.5 mL of trifluoroacetic acid and stirred overnight at room temperature, and the solvent was removed by rotary evaporation. The residue was dissolved in 0.5 mL of dichloromethane, and then precipitated by adding 5 mL of anhydrous diethyl ether. The precipitate was collected by centrifugation to obtain 1.3 mg of compound II-7, with a yield of 75%. ESI-MS: Calc.: 1070.49; Found: 1070.9 [M+H].

### Example 12: Synthesis of Compound I-12

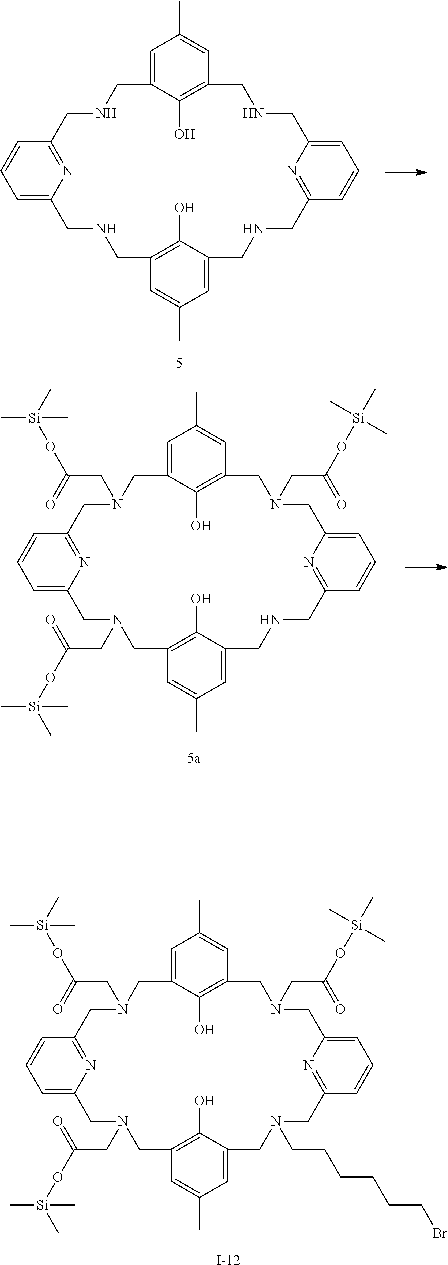

Compound 5 (100 mg, 0.93 mmol) was dispersed in 20 mL of acetonitrile, and N,N-diisopropylethylamine (187 μL, 1.07 mmol) and trimethylsilyl bromoacetate (491 mg, 2.235 mmol) were added. The mixture was stirred and reacted at room temperature for 30 min. After the reaction was complete, the solvent was removed by rotary evaporation, and the residue was purified by silica gel column chromatography to obtain 25 mg of compound 5a, with a yield of 15%.

Compound 5a (10 mg, 0.011 mmol) was dispersed in 2 mL of acetonitrile, and N,N-diisopropylethylamine (10 μL, 0.058 mmol) and 1,6-dibromohexane (4 mg, 0.016 mmol) were added. The mixture was stirred at 60° C. for 48 hours. After the reaction was complete, the solvent was removed by rotary evaporation, and the residue was purified by semi-preparative high-performance liquid chromatography to obtain 7.6 mg of compound I-12, with a yield of 64%. ESI-MS: Calc.: 1090.45/1092.44; Found: 1091.6/1093.6 [M+H].

### Example 13: Synthesis of Compound I-13

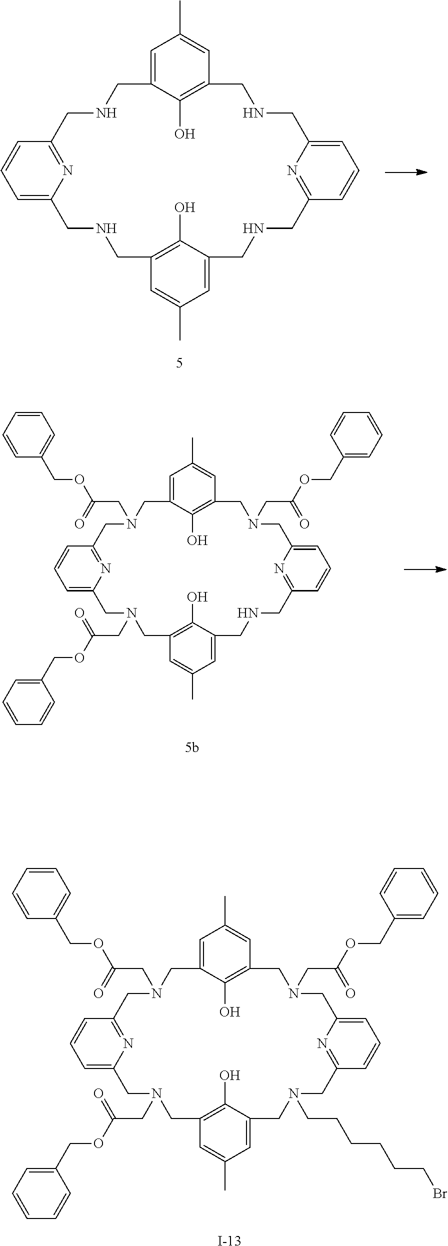

Compound 5 (100 mg, 0.93 mmol) was dispersed in 20 mL of acetonitrile, and N,N-diisopropylethylamine (187 μL, 1.07 mmol) and benzyl bromoacetate (498 mg, 2.235 mmol) were added. The mixture was stirred and reacted at room temperature for 30 min. After the reaction was complete, the solvent was removed by rotary evaporation, and the residue was purified by silica gel column chromatography to obtain 40 mg of compound 5b, with a yield of 22%.

Compound 5b (20 mg, 0.020 mmol) was dispersed in 2 mL of acetonitrile, and N,N-diisopropylethylamine (11 μL, 0.06 mmol) and 1,6-dibromohexane (8 mg, 0.030 mmol) were added. The mixture was stirred and reacted at 60° C. for 48 hours. After the reaction was complete, the solvent was removed by rotary evaporation, and the residue was purified by semi-preparative high-performance liquid chromatography to obtain 15 mg of compound I-13, with a yield of 65%. ESI-MS: Calc.: 1144.47/1046.47; Found: 1145.7/1147.7 [M+H].

### Example 14: Synthesis of Compound I-14

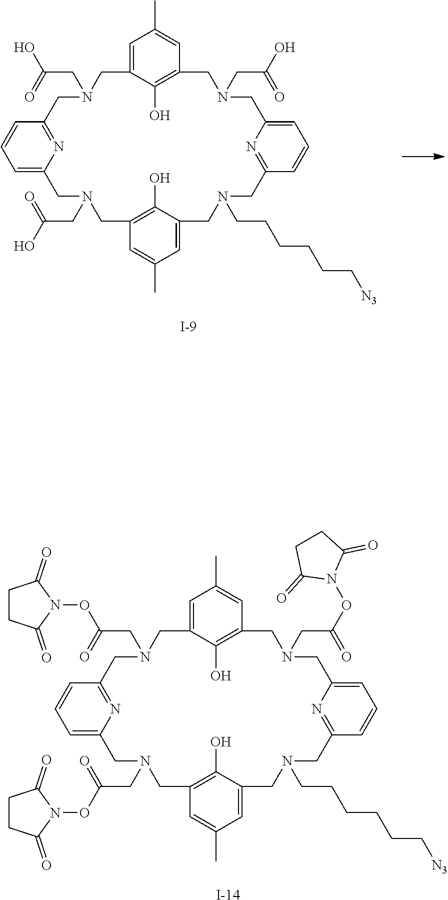

Compound I-9 (2 mg, 0.0024 mmol) was dispersed in 5 mL of acetonitrile. 1-(3-dimethylaminopropyl)-3-ethylcarbodiimide hydrochloride (1.8 mg, 0.0095 mmol) and N-hydroxysuccinimide (1.1 mg, 0.0095 mmol) were added at 0° C., and the mixture was stirred and reacted at 0° C. for 3 h. After the reaction was complete, the solvent was removed by rotary evaporation, and the residue was purified by semi-preparative high-performance liquid chromatography to obtain 800 μg of compound I-14, with a yield of 30%. ESI-MS: Calc.: 1128.47; Found: 1129.4 [M+H].

### Example 15: Synthesis of Compound I-15

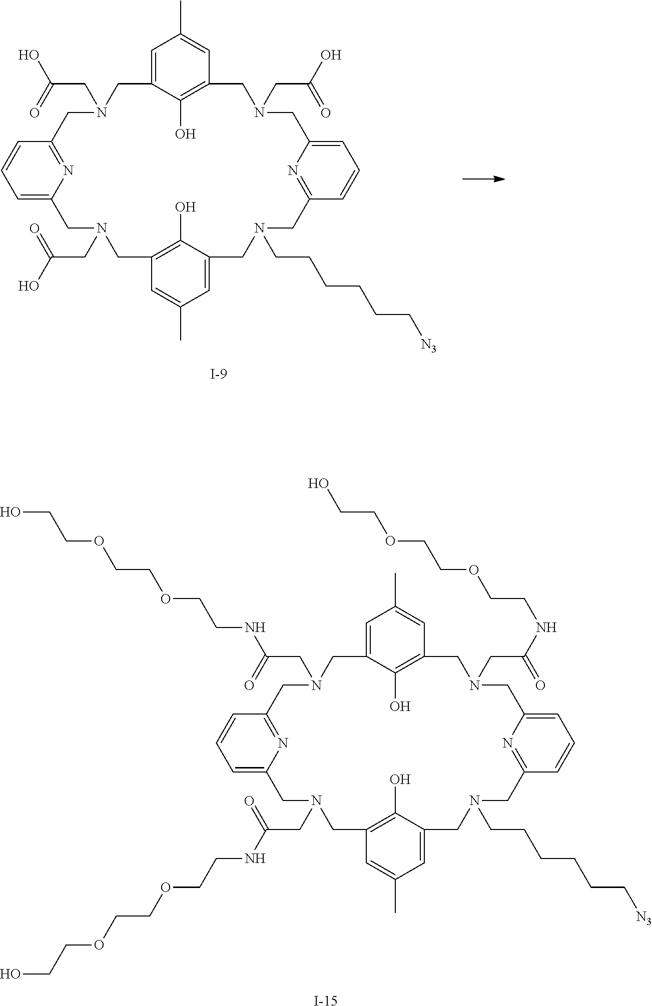

Compound I-9 (2 mg, 0.0024 mmol) was dispersed in 5 mL of acetonitrile, and N,N,N′,N′-tetramethyl-O-(7-azabenzotriazol-1-yl)uronium hexafluorophosphate (3.7 mg, 0.0096 mmol) and N,N-diisopropylethylamine (1.7 μL, 0.0096 mmol) were added, followed by addition of 2-[2-(2-aminoethoxy) ethoxy]ethanol (1.1 mg, 0.0073 mmol). The mixture was stirred at room temperature for 30 min. After the reaction was complete, the solvent was removed by rotary evaporation, and the residue was purified by silica gel column chromatography to obtain 1.9 mg of compound I-15, with a yield of 65%. ESI-MS: Calc.: 1230.70; Found: 1231.9 [M+H].

### Example 16: Synthesis of Compounds I-16/1-17

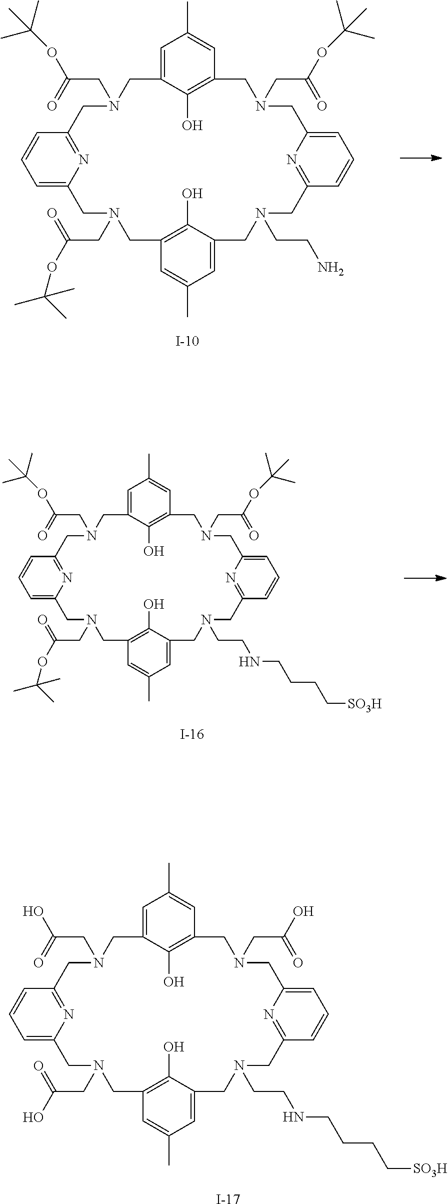

Compound I-10 (50 mg, 0.054 mmol) was added to 5 mL of tetrahydrofuran. A solution of 1,3-propene sultone (7 mg, 0.054 mmol) in tetrahydrofuran was added at 0° C. and stirred for 3 h, followed by stirring overnight at room temperature. The solvent was removed by rotary evaporation, and the residue was purified by silica gel column chromatography to obtain 37 mg of I-16, with a yield of 65%. ESI-MS: Calc.: 1059.57; Found: 1060.5 [M+H].

Compound I-16 (5 mg, 0.0047 mmol) was added to a mixed solvent of 1 mL of dichloromethane and 1 mL of trifluoroacetic acid and stirred overnight at room temperature, and the solvent was removed by rotary evaporation. The residue was dissolved in 0.5 mL of dichloromethane and precipitated in 5 mL of diethyl ether, and the precipitate was dried to obtain 3 mg of compound I-17, with a yield of 72%. ESI-MS: Calc.: 891.38; Found: 892.8 [M+H].

### Example 17: Synthesis of Compounds I-18/1-19

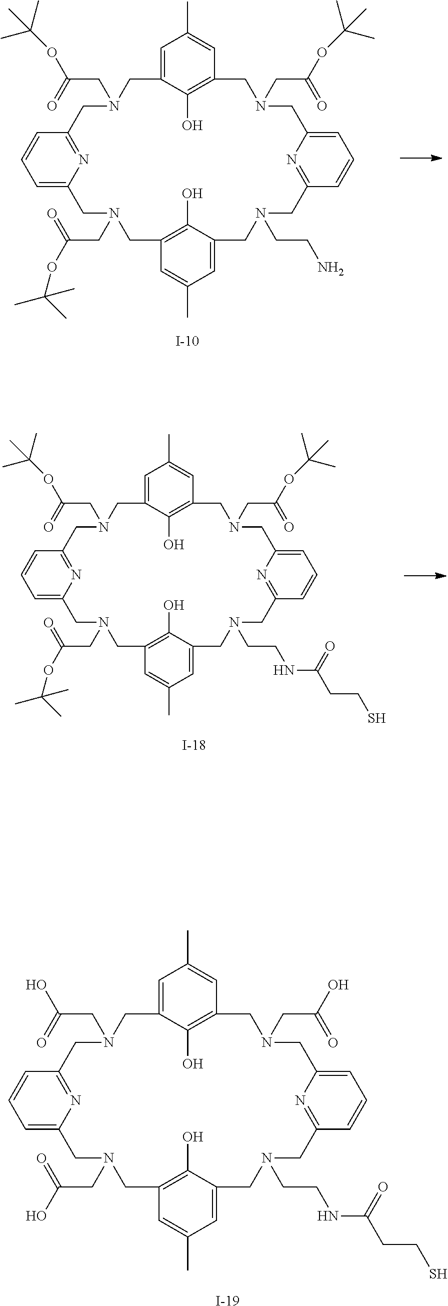

Compound I-10 (20 mg, 0.022 mmol) was dispersed in 5 mL of acetonitrile, and N,N,N′,N′-tetramethyl-O-(7-azabenzotriazol-1-yl)uronium hexafluorophosphate (14 mg, 0.036 mmol) and N,N-diisopropylethylamine (17 μL, 0.096 mmol) were added, followed by addition of 3-mercaptopropionic acid (3 mg, 0.024 mmol). The mixture was stirred and reacted at room temperature for 30 min. After the reaction was complete, the solvent was removed by rotary evaporation, and the residue was purified by semi-preparative high-performance liquid chromatography to obtain 20 mg of compound I-18, with a yield of 91%. ESI-MS: Calc.: 1011.55; Found: 1012.4 [M+H].

Compound I-18 (5 mg, 0.0049 mmol) was added to a mixed solvent of 1 mL of dichloromethane and 1 mL of trifluoroacetic acid and stirred overnight at room temperature, and the solvent was removed by rotary evaporation. The residue was dissolved in 0.5 mL of dichloromethane and precipitated in 5 mL of diethyl ether, and the precipitate was dried to obtain 3.2 mg of compound I-19, with a yield of 78%. ESI-MS: Calc.: 843.36; Found: 844.8 [M+H].

### Example 18: Synthesis of Compounds I-20/1-21/1-22

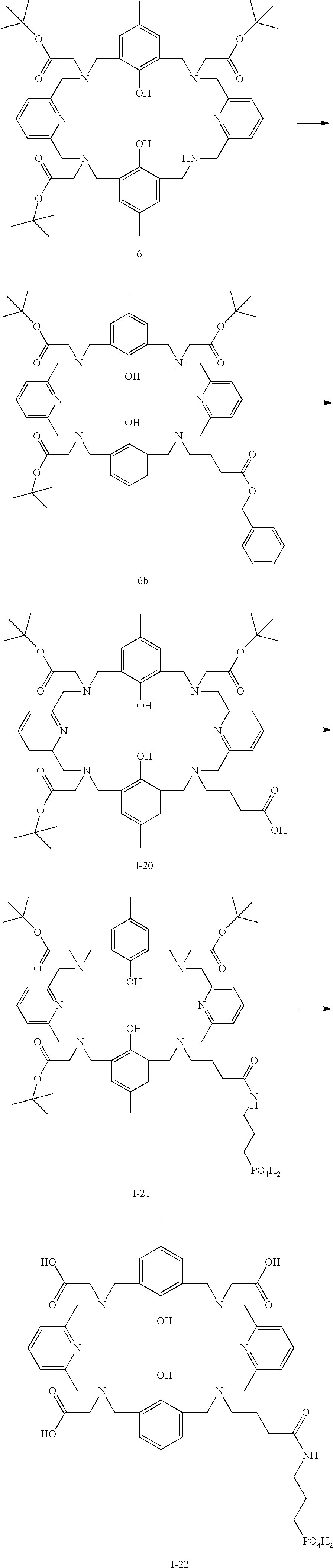

Compound 6 (100 mg, 0.114 mmol) was dispersed in 10 mL of acetonitrile, and benzyl 3-bromopropionate (55 mg, 0.228 mmol) and N,N-diisopropylethylamine (60 μL, 0.342 mmol) were added. The mixture was stirred and reacted overnight at room temperature. After the reaction was complete, the solvent was removed by rotary evaporation, and the residue was purified by silica gel column chromatography to obtain 108 mg of compound 6b.

Compound 6b (50 mg, 0.047 mmol) was dissolved in 10 mL of tetrahydrofuran, and 5 mg of 10% Pd/C was added. The reaction was carried out under hydrogen atmosphere for 12 h. The solvent was removed by rotary evaporation, and the residue was purified by silica gel column chromatography to obtain 40 mg of compound I-20, with a yield of 87%. ESI-MS: Calc.: 966.55; Found: 967.6 [M+H].

Compound I-20 (20 mg, 0.024 mmol) was dispersed in 5 mL of acetonitrile, and N,N,N′,N′-tetramethyl-O-(7-azabenzotriazol-1-yl)uronium hexafluorophosphate (14 mg, 0.036 mmol) and N,N-diisopropylethylamine (17 μL, 0.096 mmol) were added, followed by addition of 3-aminopropyl dihydrogenphosphate (4 mg, 0.024 mmol). The mixture was stirred at room temperature for 30 min. After the reaction was complete, the solvent was removed by rotary evaporation, and the residue was purified by semi-preparative high-performance liquid chromatography to obtain 10 mg of compound I-21, with a yield of 44%. ESI-MS: Calc.: 1103.57; Found: 1104.6 [M+H].

Compound I-21 (5 mg, 0.0045 mmol) was added to a mixed solvent of 1 mL of dichloromethane and 1 mL of trifluoroacetic acid and stirred overnight at room temperature, and the solvent was removed by rotary evaporation. The residue was dissolved in 0.5 mL of dichloromethane and precipitated in 5 mL of diethyl ether, and the precipitate was dried to give 3.3 mg of compound I-22 with a yield of 77%. ESI-MS: Calc.: 935.38; Found: 936.8 [M+H].

### Example 19: Synthesis of Compounds I-23/1-24

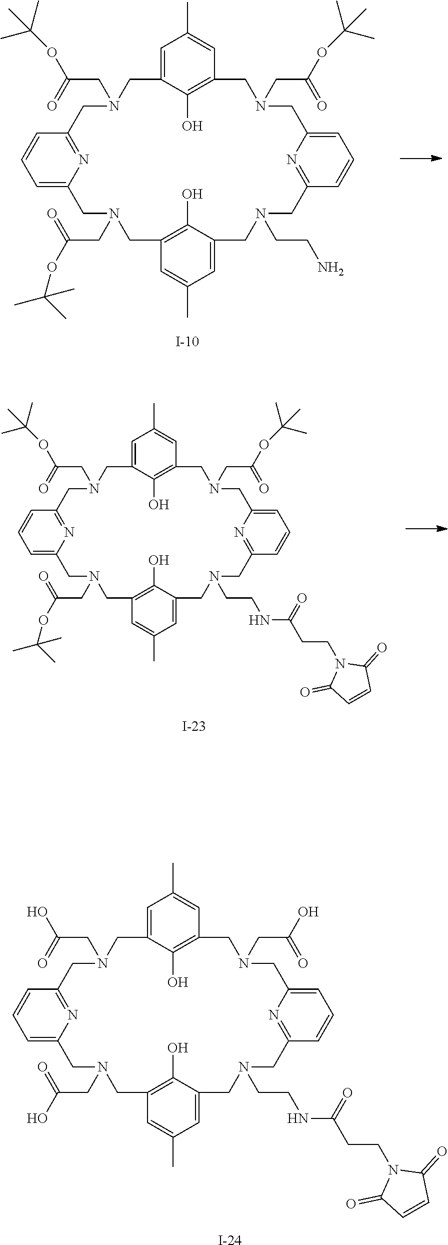

Compound I-10 (20 mg, 0.022 mmol) was dispersed in 5 mL of acetonitrile, and N,N,N′,N′-tetramethyl-O-(7-azabenzotriazol-1-yl)uronium hexafluorophosphate (14 mg, 0.036 mmol) and N,N-diisopropylethylamine (17 μL, 0.096 mmol) were added, followed by addition of 3-maleimidopropionic acid (4 mg, 0.024 mmol). The mixture was stirred and reacted at room temperature for 30 min. After the reaction was complete, the solvent was removed by rotary evaporation, and the residue was purified by semi-preparative high-performance liquid chromatography to obtain 18 mg of compound I-23, with a yield of 92%. ESI-MS: Calc.: 906.39; Found: 907.6 [M+H].

Compound I-23 (5 mg, 0.0047 mmol) was added to a mixed solvent of 1 mL of dichloromethane and 1 mL of trifluoroacetic acid and stirred overnight at room temperature, and the solvent was removed by rotary evaporation. The residue was dissolved in 0.5 mL of dichloromethane and precipitated in 5 mL of diethyl ether, and the precipitate was dried to give 3.4 mg of compound I-24, with a yield of 81%. ESI-MS: Calc.: 906.39; Found: 907.6 [M+H].

### Example 20: Synthesis of Compound I-25

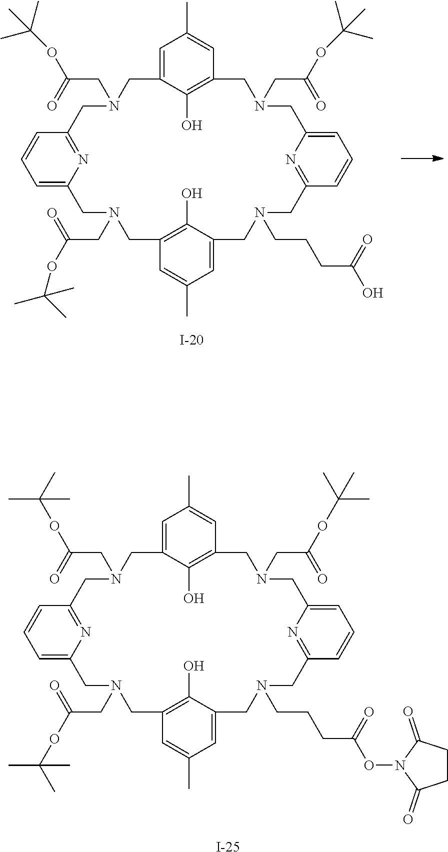

Compound 1-20 (20 mg, 0.021 mmol) was dispersed in 5 mL of acetonitrile. 1-(3-dimethylaminopropyl)-3-ethylcarbodiimide hydrochloride (7 mg, 0.036 mmol) and N-hydroxysuccinimide (4 mg, 0.036 mmol) were added at 0° C., and the mixture was stirred at 0° C. for 3 h. After the reaction was complete, the solvent was removed by rotary evaporation, and the residue was purified by semi-preparative high-performance liquid chromatography to obtain 8 mg of compound I-25, with a yield of 36%. ESI-MS: Calc.: 1063.56; Found: 1064.6 [M+H].

### Example 21: Synthesis of Compounds I-26/1-27

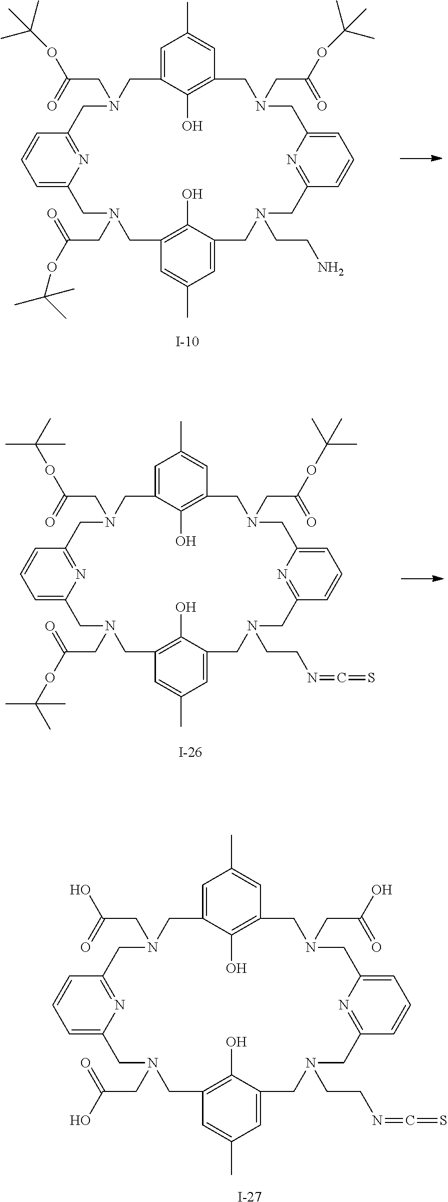

Compound I-10 (20 mg, 0.022 mmol) and sodium carbonate (38 mg, 0.36 mmol) were dispersed in 5 mL of acetone and heated under reflux for 30 min. Thiophosgene (28 mg, 0.24 mmol) was slowly added, and the mixture was stirred under reflux for another 30 min. After the reaction was complete, the solvent was removed by rotary evaporation, and the residue was purified by semi-preparative high-performance liquid chromatography to obtain 10 mg of compound I-26, with a yield of 48%. ESI-MS: Calc.: 965.51; Found: 966.6 [M+H].

Compound I-26 (5 mg, 0.0052 mmol) was added to a mixed solvent of 1 mL of dichloromethane and 1 mL of trifluoroacetic acid and stirred overnight at room temperature, and the solvent was removed by rotary evaporation. The residue was dissolved in 0.5 mL of dichloromethane and precipitated in 5 mL of diethyl ether, and the precipitate was dried to give 2.8 mg of compound I-27, with a yield of 68%. ESI-MS: Calc.: 797.32; Found: 798.6 [M+H].

### Example 22: Synthesis of Compound I-28

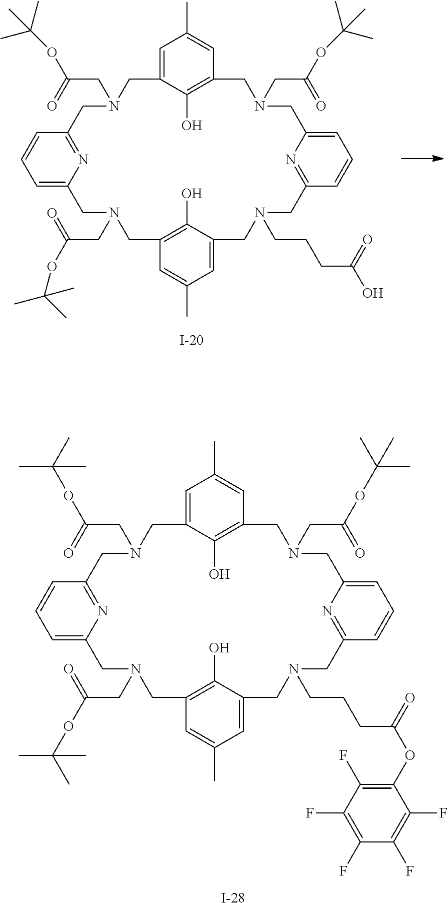

Compound I-20 (20 mg, 0.021 mmol) was dispersed in 5 mL of acetonitrile. N,N′-dicyclohexylcarbodiimide (7 mg, 0.033 mmol) and pentafluorophenol (6 mg, 0.033 mmol) were added at 0° C., and the mixture was stirred at 0° C. for 3 h. After the reaction was complete, the solvent was removed by rotary evaporation, and the residue was purified by semi-preparative high-performance liquid chromatography to obtain 8 mg of compound I-28, with a yield of 34%. ESI-MS: Calc.: 1132.53; Found: 1133.7 [M+H].

### Experimental Example

### Experimental Example 1: Labeling Rates of Compound I-5 with Different Masses Labeled with ⁶⁸Ga at 25° C. And 37° C.

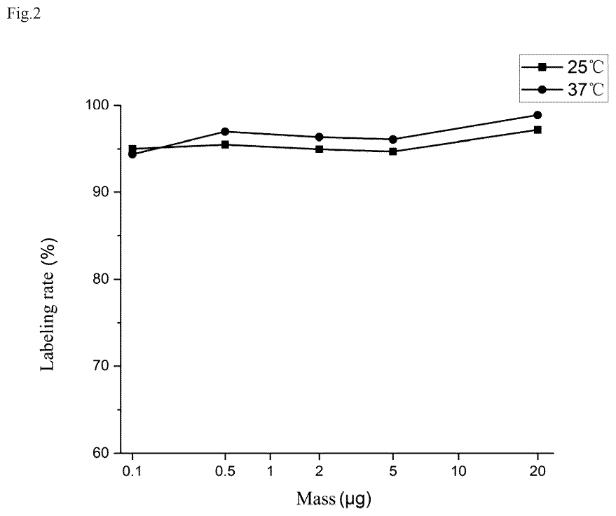

Compound I-5 was dissolved in water at a concentration of 10 μg/μL. Two groups were set, and for each group, seven samples in total with the masses of 0.1 μg, 0.5 μg, 1 μg, 2 μg, 5 μg, 10 μg, and 20 μg were added in centrifuge tubes, respectively. Then 45 μL of a 1.5M sodium acetate solution and 430 μL of a gallium chloride eluent (1 mCi) were added, mixed thoroughly, and placed at 25° C. and 37° C. for 10 min. After labeling, samples were subjected to thin-layer chromatography, and the labeling rate was calculated after instrument analysis. The results were shown in FIG. 2. The results showed that, within the experimental concentration range, the labeling rates of compound I-5 were over 90% at both 25° C. and 37° C.; and at the concentrations of 0.5 μg or more, the labeling efficiency was higher at 37° C. than those at 25° C.; at 37° C., the labeling rate of 20 μg Ie could approach 100%, indicating that it could be used in radiological experiments without purification.

### Example 2: PET/CT Images of the Complex ⁶⁸Ga-II-2 (Compound II-2 Labeled with ⁶⁸Ga) in U87 Tumor Mice

20 μg of compound II-2 was dissolved in water, and 45 μL of this solution was added to a mixed solution of 45 μL of 1.5M sodium acetate solution and 430 μL of gallium chloride eluent (1 mCi) and thoroughly mixed. The mixture was placed at 37° C. for 10 min. After labeling, ⁶⁸Ga-II-2 was obtained. A sample was then subjected to thin-layer chromatography, and the labeling rate was calculated after instrument analysis. The labeling rate at 37° C. was 99%, which is sufficient for direct in vivo imaging in mice.

The mice used in the experiment were purchased with approval from the Shanghai Laboratory Animal Center, and the animal experiments were conducted in accordance with the guidelines of the Institutional Animal Care and Use Committee (IACUC) of the Shanghai Institute of Materia Medica, Chinese Academy of Sciences. Six-week-old female Balb/c mice were inoculated subcutaneously with tumors in their hind legs. After tumor formation, the diameter of the tumor was measured using a caliper. The tumor volume was calculated using the equation: tumor volume (V)=(length×width²)/2. The mice were used after the tumor reached 150 mm³.

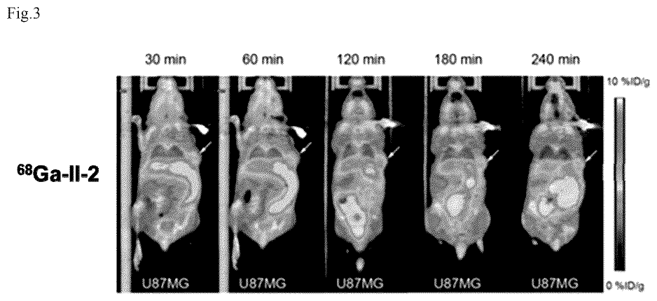

The ⁶⁸Ga-II-2 complex was injected via the tail vein into U87 tumor-bearing mice for PET/CT in vivo imaging. PET/CT images were acquired 30 min, 1 h, 2 h, 3 h, and 4 h after injection to observe the distribution of the probe in the mice and its enrichment in the tumor region. The results are shown in FIG. 3.

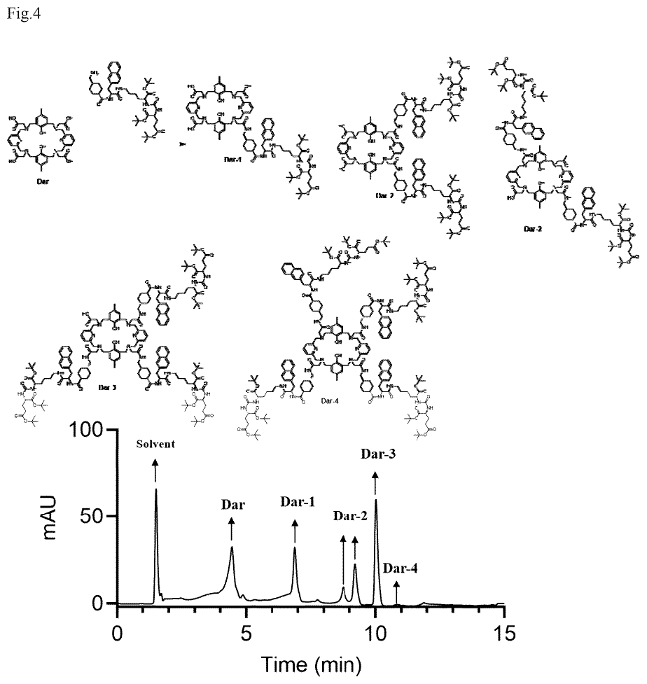

FIG. 4 shows a high performance liquid chromatogram of a reaction solution of the existing macrocyclic chelating agent Dar conjugated with PSMA. It can be seen that Dar has no selectivity with multiple conjugating sites conjugated at the same time. In addition to the target product (Dar-1), there are other peaks (Dar-2, Dar-3, Dar-4), resulting in low yield and difficult purification.

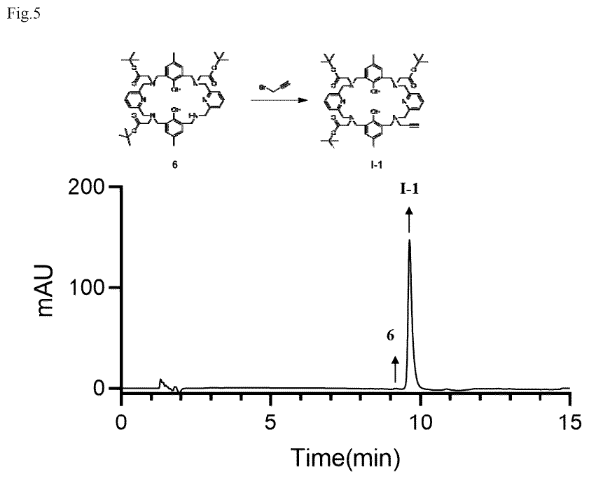

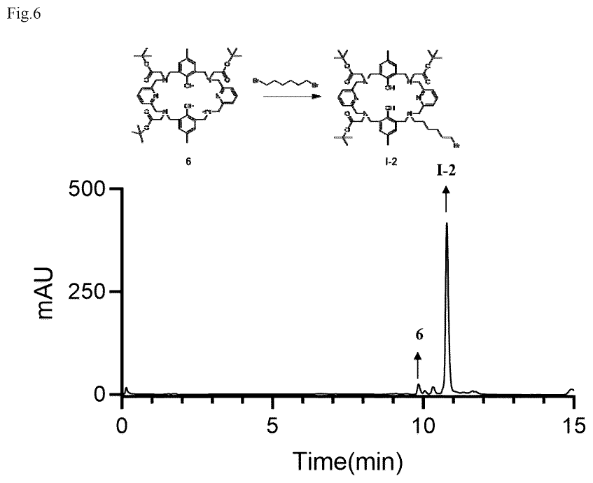

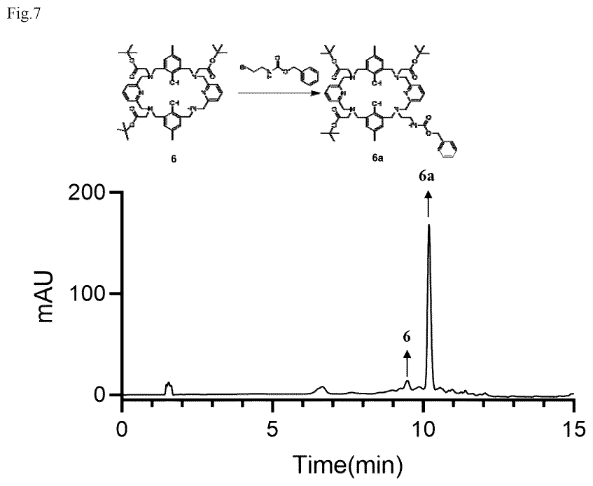

As can be seen from FIGS. 5-7, the macrocyclic chelating agent of the present invention is specific for the conjugating site, and the product is mainly a monosubstituted product, with high yield and easy purification.

The above examples are merely illustrative of the technical solutions of the present invention and are not intended to limit it. Although the present invention has been described in detail with reference to the foregoing examples, those skilled in the art should understand that modifications can be made to the technical solutions described in the foregoing examples, or equivalent substitutions can be made to some or all of the technical features therein, without departing from the spirit and substance defined by the claims of the present invention; and such modifications or substitutions are still within the scope defined by the claims of the present invention.

1. A compound of formula I, or a precursor compound, isotopic compound, salt, or hydrate thereof, 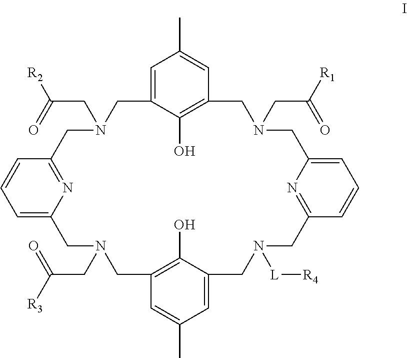
wherein,
R₁, R₂, and R₃ are each independently selected from —OH, —ORₐ, oxygen-containing long-chain polymer group or derivative groups thereof;
Rₐ is selected from substituted or unsubstituted C₁-C₁₂ alkyl, substituted or unsubstituted C₁-C₁₂ silyl, substituted or unsubstituted C₃-C₁₆ cycloalkyl, substituted or unsubstituted 3- to 16-membered heterocyclyl, substituted or unsubstituted C₆-C₁₆ aryl, and substituted or unsubstituted 5- to 16-membered heteroaryl, wherein the substituted refers to being substituted by one or more substituents selected from halogen, C₁-C₆ alkyl, C₁-C₆ alkoxy, oxo, and amino;
L is a linker; and
R₄ is an active group capable of reacting with a target molecule.

2. The compound of formula I, or a precursor compound, isotopic compound, salt, or hydrate thereof according to claim 1, wherein,
R₁, R₂, and R₃ are each independently selected from —OH, —ORₐ, PEG or derivative groups thereof; Rₐ is selected from substituted or unsubstituted C₁-C₆ alkyl, substituted or unsubstituted C₁-C₆ silyl, substituted or unsubstituted C₆-C₁₂ aryl, and substituted or unsubstituted 5-10 heteroaryl;
wherein Rₐ is selected from tert-butyl, trimethylsilyl (Si(CH₃)₃), benzyl, and N-succinimidyl;
wherein the PEG or derivative groups thereof are selected from 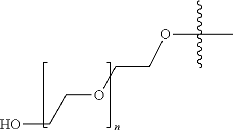
(n is an integer selected from 0 to 10) and 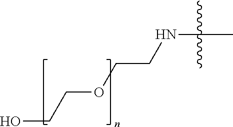
(n is an integer selected from 0 to 10).

3. The compound of formula I, or a precursor compound, isotopic compound, salt, or hydrate thereof according to claim 1, wherein,
R₄ is any one selected from C₂-C₆ alkynyl, azido, halogen, formyl, hydroxyl, amino, carboxyl, sulfo, phosphate, mercapto, sulfinamido, isothiocyanato (—N═C═S), pentafluorophenyl, maleimido, and N-succinimidyl.

4. The compound of formula I, or a precursor compound, isotopic compound, salt, or hydrate thereof according to claim 1, wherein,
L is selected from C₁-C₁₂ alkylene, divalent long-chain polymer groups or derivative groups thereof, divalent amino acid groups and derivative groups thereof, divalent polypeptide groups, and bifunctional linkers,
wherein the divalent long-chain polymer groups or derivative groups thereof are divalent PEG or derivative groups thereof selected from 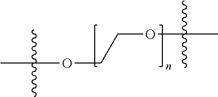
(n is an integer selected from 0 to 10) and 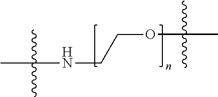
(n is an integer selected from 0 to 10);
wherein the bifunctional linkers are selected from the following structures: 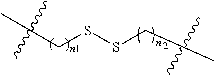
(n1 and n2 are each an integer from 0 to 10), 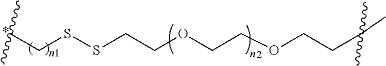
(n1 and n2 are each an integer from 0 to 5), 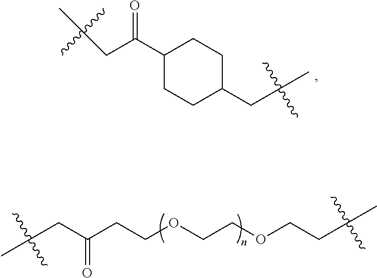
(n1 is an integer from 0 to 5), 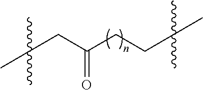
(n is an integer from 0 to 10), 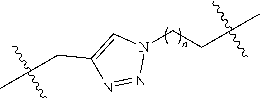
(n is an integer from 0 to 10), 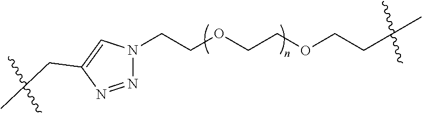
(n is an integer from 0 to 5), 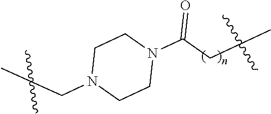
(n is an integer from 0 to 5), 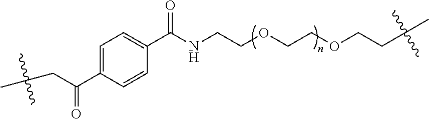
(n is an integer from 0 to 5), 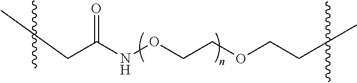
(n is an integer from 0 to 5), 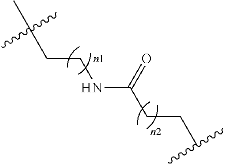
(n1 and n2 are each an integer from 0 to 5), 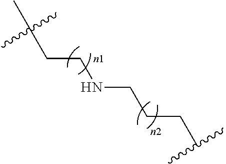
(n1 and n2 are each an integer from 0 to 5), 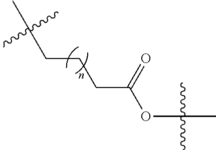
(n is an integer from 0 to 5).

5. The compound of formula I, or a precursor compound, isotopic compound, salt, or hydrate thereof according to claim 1, wherein the compound of Formula I is selected from the following compounds:

| No. | Chemical Structure |
|---|---|
| I-1 | 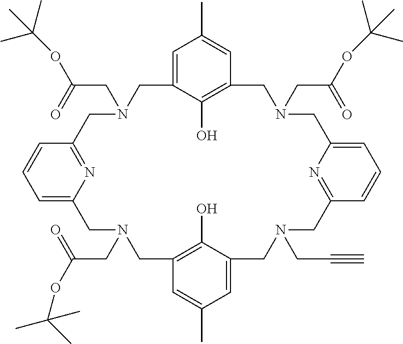 [SMILES: C#CCN1Cc2cccc(n2)CN(CC(=O)OC(C)(C)C)Cc2cc(C)cc(c2O)CN(CC(=O)OC(C)(C)C)Cc2cccc(n2)CN(CC(=O)OC(C)(C)C)Cc2cc(C)cc(c2O)C1] |
| I-2 | 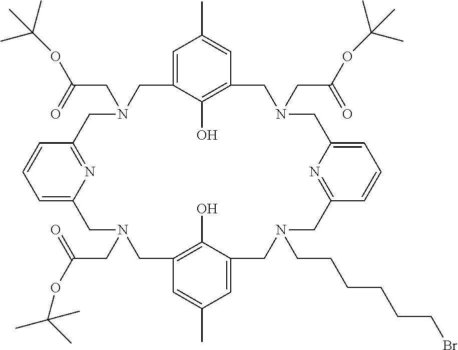 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCCBr)C2] |
| I-3 | 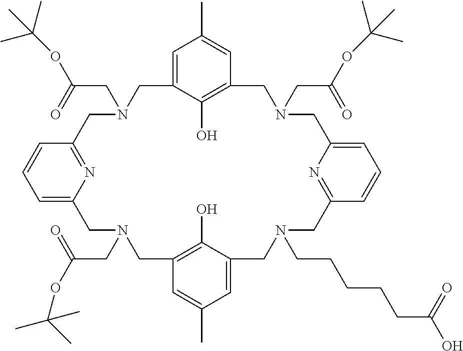 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCC(=O)O)C2] |
| I-4 | 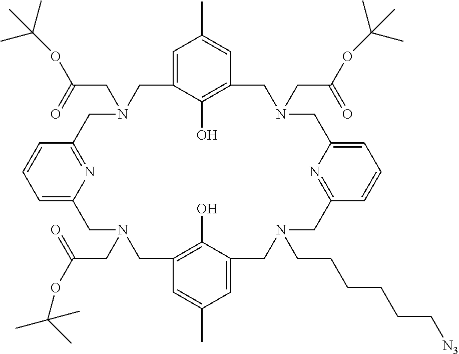 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCCN=[N+]=[N-])C2] |
| I-5 | 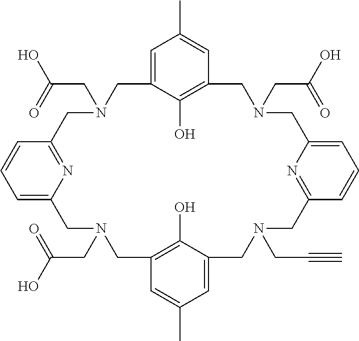 [SMILES: C#CCN1Cc2cccc(n2)CN(CC(=O)O)Cc2cc(C)cc(c2O)CN(CC(=O)O)Cc2cccc(n2)CN(CC(=O)O)Cc2cc(C)cc(c2O)C1] |
| I-6 | 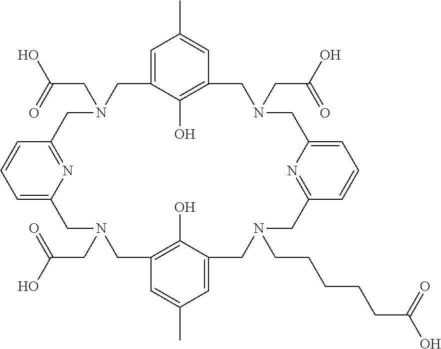 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCC(=O)O)C2] |
| I-7 | 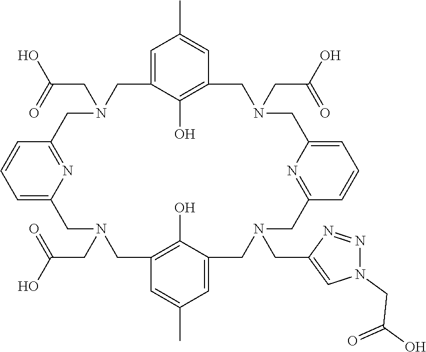 [SMILES: Cc1cc2c(O)c(c1)CN(CC(=O)O)Cc1cccc(n1)CN(Cc1cn(CC(=O)O)nn1)Cc1cc(C)cc(c1O)CN(CC(=O)O)Cc1cccc(n1)CN(CC(=O)O)C2] |
| I-8 | 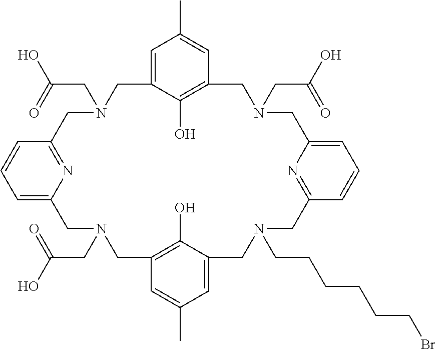 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCCBr)C2] |
| I-9 | 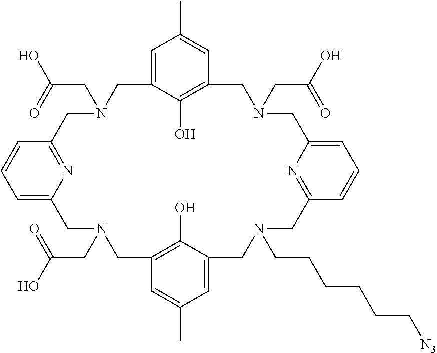 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCCN=[N+]=[N-])C2] |
| I-10 | 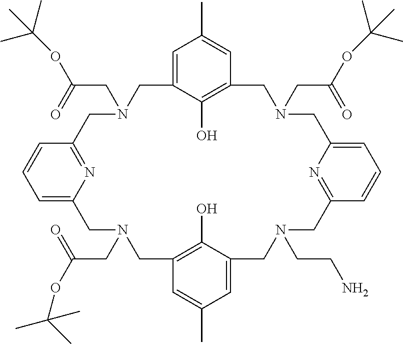 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCN)C2] |
| I-11 | 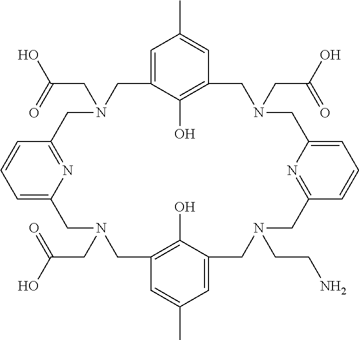 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCN)C2] |
| I-12 | 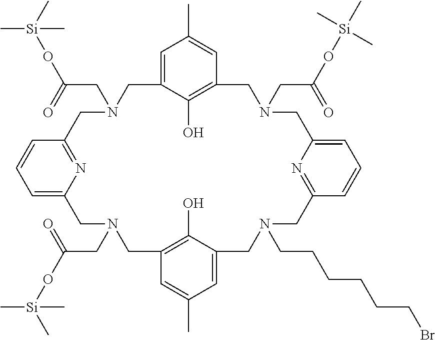 [SMILES: Cc1cc2c(O)c(c1)CN(CC(=O)O[Si](C)(C)C)Cc1cccc(n1)CN(CC(=O)O[Si](C)(C)C)Cc1cc(C)cc(c1O)CN(CC(=O)O[Si](C)(C)C)Cc1cccc(n1)CN(CCCCCCBr)C2] |
| I-13 | 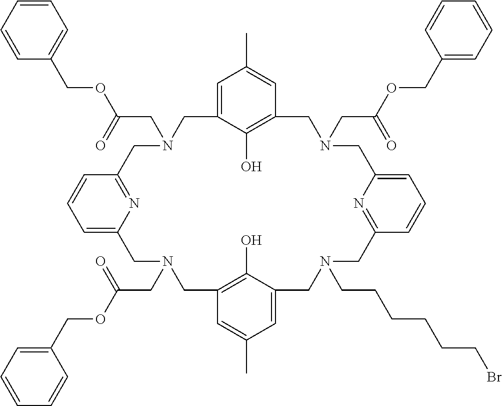 [SMILES: Cc1cc2c(O)c(c1)CN(CC(=O)OCc1ccccc1)Cc1cccc(n1)CN(CC(=O)OCc1ccccc1)Cc1cc(C)cc(c1O)CN(CC(=O)OCc1ccccc1)Cc1cccc(n1)CN(CCCCCCBr)C2] |
| I-14 | 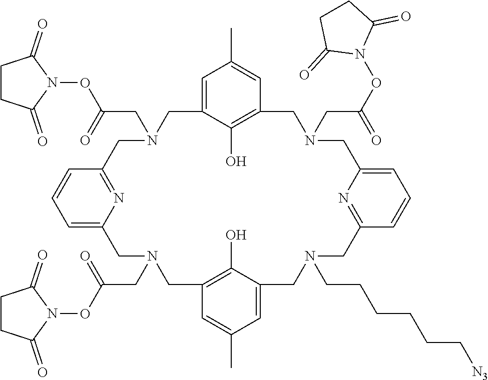 [SMILES: Cc1cc2c(O)c(c1)CN(CC(=O)ON1C(=O)CCC1=O)Cc1cccc(n1)CN(CC(=O)ON1C(=O)CCC1=O)Cc1cc(C)cc(c1O)CN(CC(=O)ON1C(=O)CCC1=O)Cc1cccc(n1)CN(CCCCCCN=[N+]=[N-])C2] |
| I-15 | 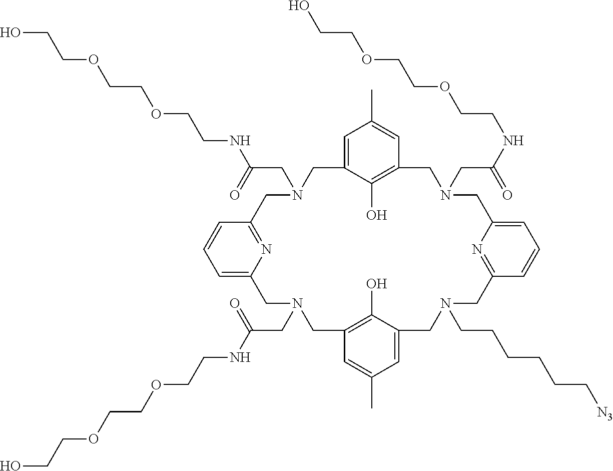 [SMILES: Cc1cc2c(O)c(c1)CN(CC(=O)NCCOCCOCCO)Cc1cccc(n1)CN(CC(=O)NCCOCCOCCO)Cc1cc(C)cc(c1O)CN(CC(=O)NCCOCCOCCO)Cc1cccc(n1)CN(CCCCCCN=[N+]=[N-])C2] |
| I-16 | 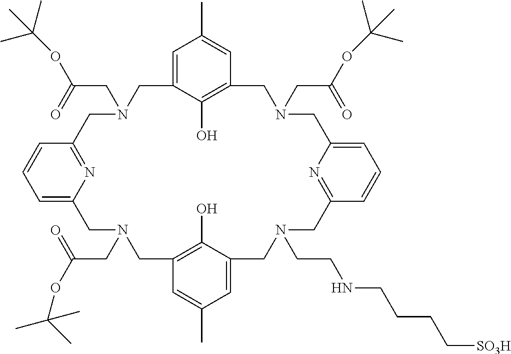 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCNCCCCS(=O)(=O)O)C2] |
| I-17 | 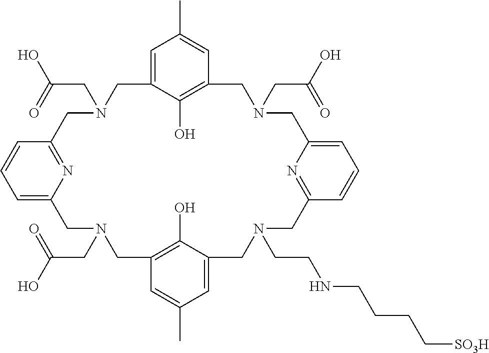 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCNCCCCS(=O)(=O)O)C2] |
| I-18 | 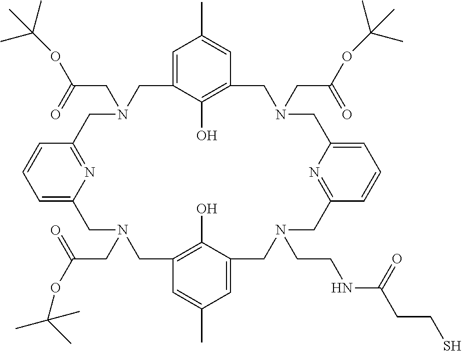 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCNC(=O)CCS)C2] |
| I-19 | 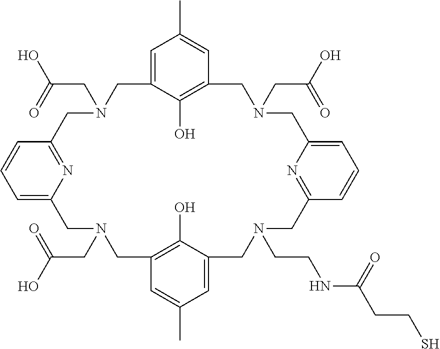 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCNC(=O)CCS)C2] |
| I-20 | 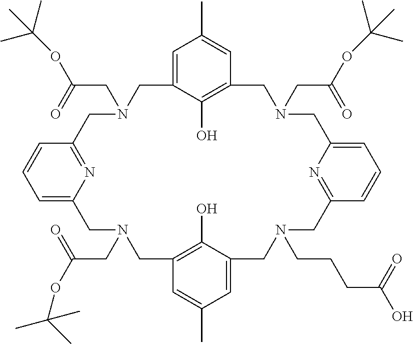 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCC(=O)O)C2] |
| I-21 | 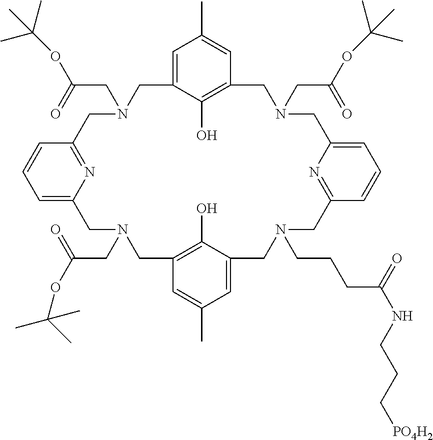 |
| I-22 | 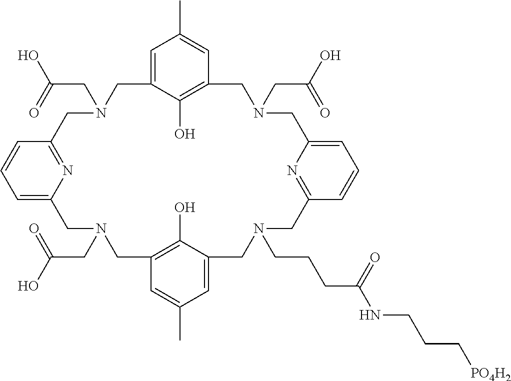 |
| I-23 | 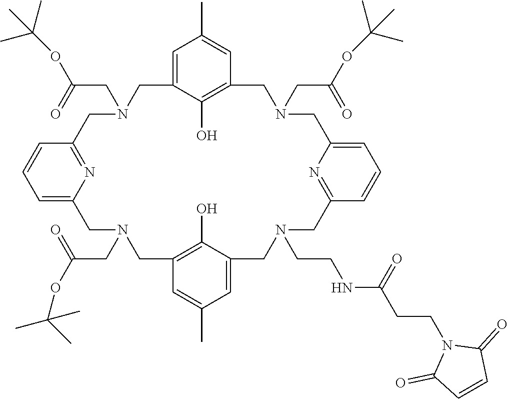 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCNC(=O)CCN1C(=O)C=CC1=O)C2] |
| I-24 | 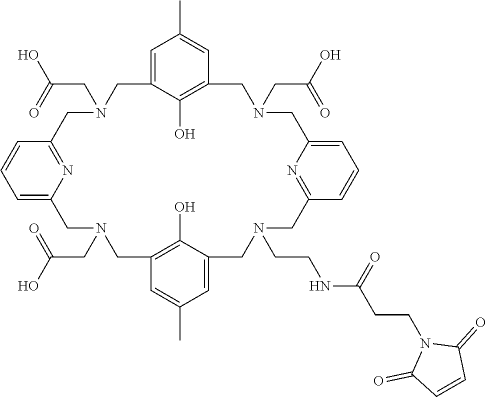 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCNC(=O)CCN1C(=O)C=CC1=O)C2] |
| I-25 | 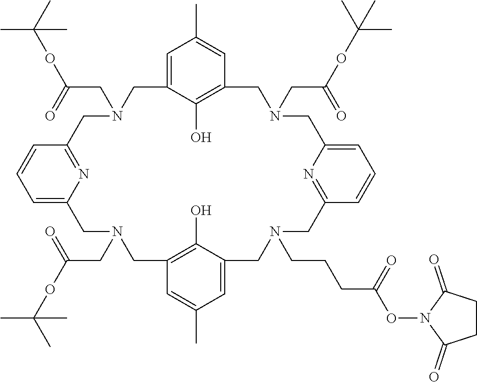 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCC(=O)ON1C(=O)CCC1=O)C2] |
| I-26 | 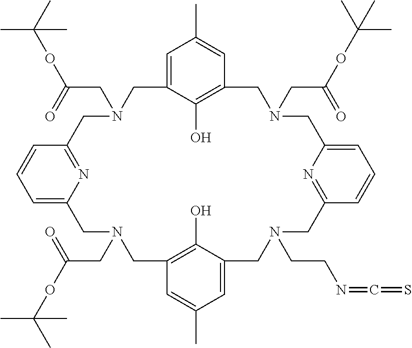 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCN=C=S)C2] |
| I-27 | 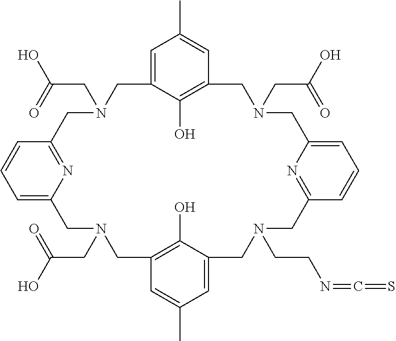 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCN=C=S)C2] |
| I-28 | 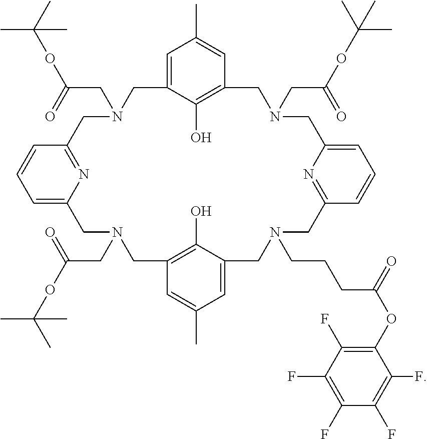 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCC(=O)Oc1c(F)c(F)c(F)c(F)c1F)C2] |

6. A method for preparing a nuclide probe, using the compound of Formula I, or a precursor compound, isotopic compound, salt or hydrate thereof according to claim 1 as a macrocyclic chelating agent capable of site-specific modification.

7. A macrocyclic chelating agent conjugate formed by conjugating the compound of Formula I, or a precursor compound, isotopic compound, salt or hydrate thereof according to claim 1, to a target molecule,
wherein the target molecule is any one selected from antibodies, peptides, saccharides, nucleic acids, small molecule drugs, optical dyes, organic nanomaterials, or fragments thereof or derivatives thereof,
wherein the antibody is selected from molecules containing a Fv fragment, and further selected from monoclonal antibodies, bispecific antibodies, and single-chain Fv fragments,
wherein the peptide is any one selected from octreotide, c(RGDyk), c(RGDfk), c(RADyk), folic acid, PSMA-617, and PSMA-11,
wherein the saccharide is any one selected from glucose, galactose, fructose, arabinose, rhamnose, ribose, mannose, lactose, maltose, amygdalin, and cyclodextrin;
wherein the nucleic acid is any one selected from deoxyribonucleic acid (DNA), ribonucleic acid (RNA), aptamers, and DNA/RNA hybrids,
wherein the small molecule drug is any one selected from afatinib, axitinib, bleomycin, camptothecin, capecitabine, dabrafenib, dasatinib, docetaxel, doxorubicin, and paclitaxel,
wherein the optical dye is any one selected from CH-1055, CH-4T, FD-1080, ICG, BODIPY, Rhodamine, IRdye800CW, and IR-1061,
wherein the organic nanomaterial is a liposome; and
wherein the macrocyclic chelating agent conjugate is selected from the following structures:

| No. | Chemical Structure |
|---|---|
| II-1 | 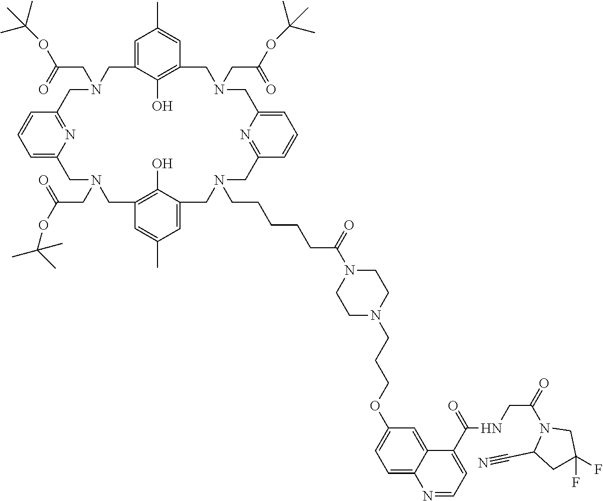 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCC(=O)N1CCN(CCCOc3ccc4nccc(C(=O)NCC(=O)N5CC(F)(F)CC5C#N)c4c3)CC1)C2] |
| II-2 | 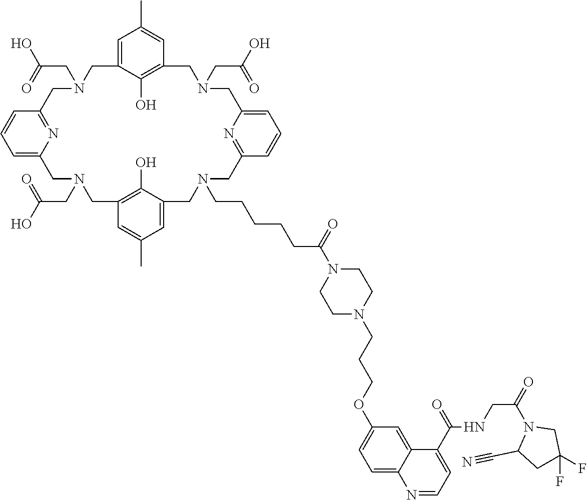 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCC(=O)N1CCN(CCCOc3ccc4nccc(C(=O)NCC(=O)N5CC(F)(F)CC5C#N)c4c3)CC1)C2] |
| II-3 | 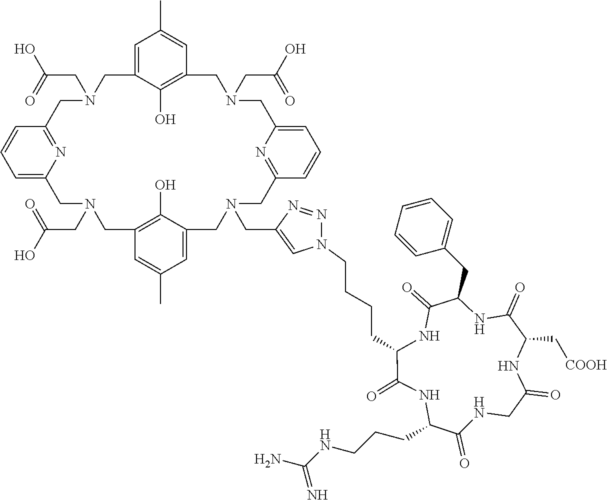 [SMILES: Cc1cc2c(O)c(c1)CN(CC(=O)O)Cc1cccc(n1)CN(Cc1cn(CCCC[C@@H]3NC(=O)[C@@H](Cc4ccccc4)NC(=O)[C@H](CC(=O)O)NC(=O)CNC(=O)[C@H](CCCNC(=N)N)NC3=O)nn1)Cc1cc(C)cc(c1O)CN(CC(=O)O)Cc1cccc(n1)CN(CC(=O)O)C2] |
| II-4 | 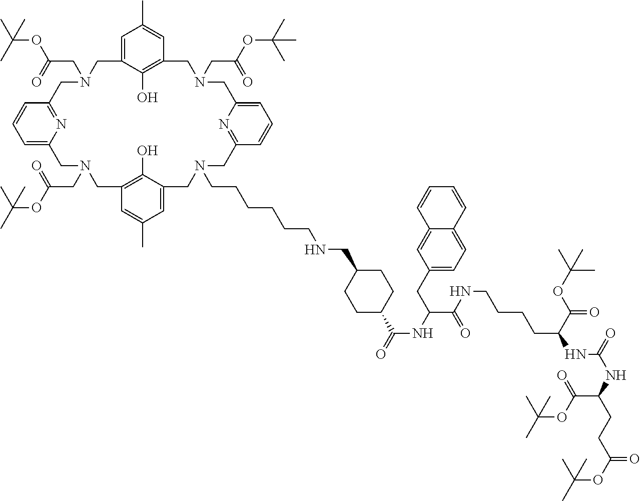 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCCCCCNC[C@H]1CC[C@H](C(=O)NC(Cc3ccc4ccccc4c3)C(=O)NCCCC[C@H](NC(=O)N[C@@H](CCC(=O)OC(C)(C)C)C(=O)OC(C)(C)C)C(=O)OC(C)(C)C)CC1)C2] |
| II-5 | 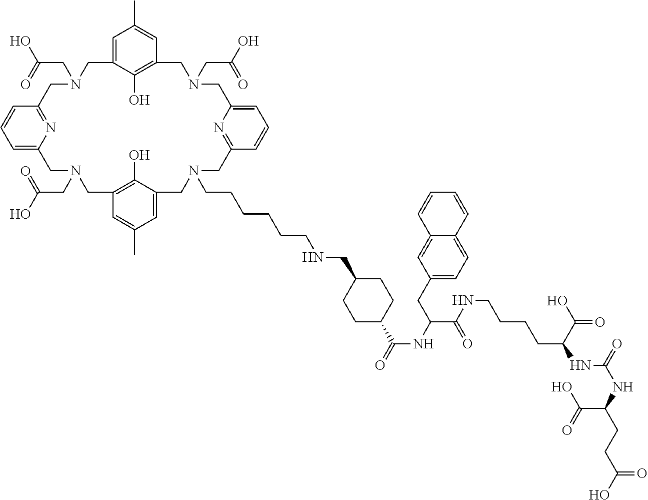 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCCCCCNC[C@H]1CC[C@H](C(=O)NC(Cc3ccc4ccccc4c3)C(=O)NCCCC[C@H](NC(=O)N[C@@H](CCC(=O)O)C(=O)O)C(=O)O)CC1)C2] |
| II-6 | 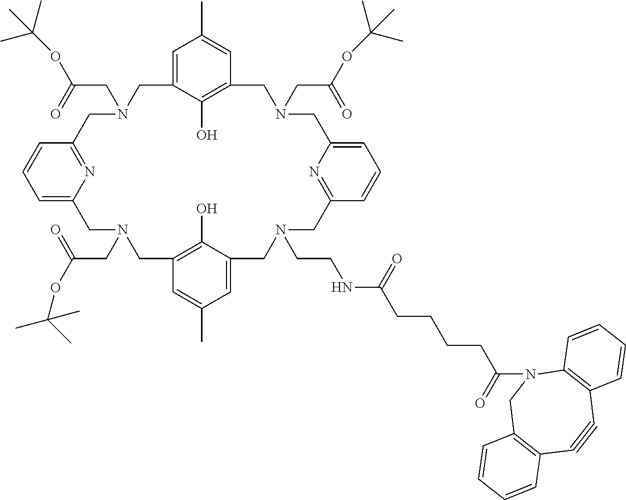 [SMILES: Cc1cc2c(O)c(c1)CN(CC(=O)OC(C)(C)C)Cc1cccc(n1)CN(CC(=O)OC(C)(C)C)Cc1cc(C)cc(c1O)CN(CC(=O)OC(C)(C)C)Cc1cccc(n1)CN(CCNC(=O)CCCCC(=O)N1Cc3ccccc3C#Cc3ccccc31)C2] |
| II-7 | 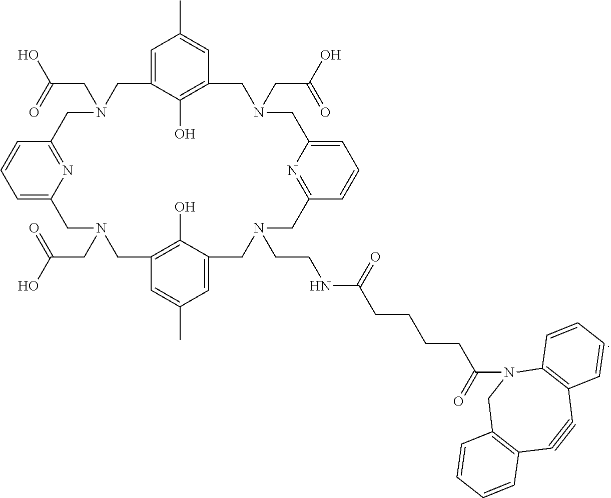 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CCNC(=O)CCCCC(=O)N1Cc3ccccc3C#Cc3ccccc31)C2] |

8. A nuclide probe, which is one or more of the macrocyclic chelating agent conjugate according to claim 7 labelled with a nuclide,
wherein the nuclide is a radioactive nuclide or a non-radioactive nuclide,
wherein the radioactive nuclide is any one selected from ⁹⁰Y, ¹³¹I, ¹⁵³Sm, ¹⁷⁷Lu, ¹⁸⁶Re, ¹⁸⁸ Re, ²¹¹At, ²¹²Pb, ²²³Ra, ²²⁵Ac, Al[¹⁸F], ⁵¹Mn, ^{52m}Mn, ^{52g}Mn, ⁶⁴Cu, ⁶⁷Cu, ⁶⁷Ga, ⁶⁸Ga, ⁸⁶Y, ⁸⁹Zr, ^{99m}Tc, ¹¹¹In, ¹²³I, ¹²⁴I, ¹²⁵I, ¹⁶⁶Ho, ²¹³Bi, ^{227Th}, and
wherein the non-radioactive nuclide is any one selected from Mn, Si, Pt, Nd, Gd, Yb, Er, Cu, Y, Fe, Tm.

9. A molecular imaging method comprising administrating to a subject in need thereof an effective amount of the nuclide probe according to claim 8,
wherein the molecular imaging method includes PET imaging, SPECT imaging, optical imaging, MRI imaging, and multimodal imaging formed by their combination with each other.

10. A molecular imaging diagnostic method comprising administrating to a subject in need thereof an effective amount of the nuclide probe according to claim 8 as a molecular imaging diagnostic agent.

11. A method for treating a tumor comprising administrating to a subject in need thereof an effective amount of the nuclide probe according to claim 8.
